(19) United States
(12) Patent Application Publication
(10) Pub. No.: US 2003/0027207 A1
(43) Pub. Date: Feb. 6, 2003

(54) Anti-platelet binding proteins and polymer conjugates containing the same

(76) Inventor: David Ray Filpula, Piscataway, NJ (US)

Correspondence Address: ROBERTS & MERCANTI, L.L.P., EDC II, Suite 203, 105 Lock Street, Newark, NJ, 07103, US

(21) Appl. No.: 09/794,189

(22) Filed: Feb. 27, 2001

Related U.S. Application Data
(60) Provisional application No. 60/185,628, filed on Feb. 29, 2000.

(51) Int. Cl. G01N 33/53; C12Q 1/70; C07H 21/04; C12P 21/02; C12N 5/06; C07K 16/34; C07K 16/36

(52) U.S. Cl. 435/7.1; 435/5; 435/69.1; 435/325; 435/320.1; 536/23.2; 435/69.3; 530/388.7

(57) ABSTRACT

The invention provides for anti-platelet binding proteins, and corresponding nucleic acids encoding the binding proteins, that are isolated, for example, from a phage display library by an in vitro selection process. The in vitro selection process involves screening a diverse human antibody variable domain expression library against at least one human platelet antigen to identify clones expressing single-chain antigen-binding proteins useful for inhibiting platelet aggegation and thrombosis, both in vitro and in vivo, as well as in both in vitro and in vivo assays and diagnostic procedures. Anti-platelet binding proteins conjugated to substantially non-antigenic polymers are also provided.

This application hereby claims priority from Provisional U.S. Patent Application Ser. No. 60/185,628, filed on Feb. 29, 2000, incorporated by reference herein in its entirety.

### FIELD OF THE INVENTION

The invention pertains to novel anti-platelet antigen-binding proteins, including antibodies, antibody fragments and single-chain antigen-binding proteins or SCA® proteins, that inhibit platelet aggregation and/or the interaction of fibrinogen with platelets and/or inhibit thrombus formation, in vitro and in vivo, and to methods of preparing and using such anti-platelet antigen-binding proteins. The invention also pertains to conjugates of anti-platelet antigen-binding proteins to substantially non-antigenic polymers.

### BACKGROUND OF THE INVENTION

Hemostasis is the complex process wherein the circulatory systems of humans and other vertebrates reacts to blood leakage or bleeding from injured blood vessels or tissues. Hemostasis results from the coordinated activity of vascular, platelet, and plasma factors. However, although there are regulatory mechanisms that limit the accumulation of platelets and fibrin in the area of injury, under certain conditions defects in the components of the hemostatic mechanism and/or damage to blood vessel walls can lead to excessive bleeding or potentially lethal localized or disseminated thrombosis.

Thrombosis is a pathological process in which a platelet aggregate and/or a fibrin clot forms in a location that can harm the other tissues or organs in the body, e.g., by occluding blood flow to one or more vital tissues or organs of the body. If a blood vessel is injured, platelets first adhere to macromolecules in the subendothelium of the injured blood vessel. As the platelets aggregate, they form a primary hemostatic plug. Platelet aggregation activates plasma coagulation factors, resulting in a cascade of events, including the conversion of fibrinogen to fibrin, which reinforce the platelet aggregate. A mass that results from this process is a thrombus, and a thrombus (plural is thrombi) can form within the cardiovascular system on denuded and/or injured endovascular or prosthetic flow surfaces. Thrombi are composed of insoluble fibrin, deposited platelets, accumulating white blood cells, and entrapped erythrocytes in variable flow-dependent patterns.

Patients with symptomatic atherosclerosis are at significant risk of stroke, myocardial infarction (“M.I.”), and peripheral artery occlusion, which primarily develop at sites of preexisting stenosis. If an atherosclerotic plaque ruptures and exposes tissue factor-rich plaque contents to blood, this can initiate thrombin-mediated, platelet-rich thrombus formation. This is particularly damaging when a thrombus occurs in an artery perfusing an organ without substantial collateral circulation, such as the heart, brain and kidneys. Thrombus formation at an atherosclerotic plaque in an artery perfusing such critical tissues can occlude the blood vessel and damage the tissue or organ supplied by that vessel. This can also result from a ruptured plaque that throws off fragments or particles to be carried downstream to narrower blood vessels supplying other parts of the body, occluding circulation to tissues distal to the occluding thrombus.

Patients who have received, or who are about to receive, invasive treatment for coronary artery occlusion, and similar medical conditions, are at even greater risk for thrombus formation. For example, patients at high risk for thrombus formation are those undergoing or about to undergo percutaneous coronary intervention for the prevention of cardiac ischemic complications, patients with, e.g., unstable angina not responding to conventional medical therapy, and who will be undergoing percutaneous coronary intervention within about 24 hours, as well as in patients at high risk for abrupt closure of a treated coronary vessel, e.g., after surgical coronary artery bypass or endoscopic treatment.

Current agents used to treat or prevent thrombosis and related conditions fall into a number of classes, such as anticoagulants, thrombolytics and anti-platelet agents. For example, anticoagulants include, e.g., glycosaminoglycans such as heparin, and the oral anticoagulants, eg., coumarin derivatives, such as 4-hydroxycoumarin, indan-1,3-dione and a variety of derivatives of these compounds. Thrombolytic agents are more recently introduced, and include enzymes such as plasminogen, streptokinase, tissue plasminogen activator (t-PA), and urokinase. Unfortunately, there is a risk of re-occlusion when using thrombolytic agents, which can result in further damage to tissues perfused by the occluded artery.

Current anti-platelet drugs include aspirin, that is used therapeutically or prophylactically, alone or in combination with other agents, e.g., with a vasodilator, dipyridamole, which also interacts with platelet adenyl cyclase. Another current drug is ticlopidine, a thienopyridine said to interact with platelet glycoprotein IIb/IIIa (“GPIIb/IIIa”) in an unknown way to inhibit the binding of fibrinogen to activated platelets. GPIIb/IIIa is a dimeric protein present on platelet surfaces that is described in the literature as a member of the integrin family of adhesion receptors that binds fibrinogen. In particular GPIIb/IIIa is considered to be the major platelet surface receptor involved in platelet aggregation.

Thus, the art has considered GPIIb/IIIa to be a target for inhibiting thrombosis, although an optimal agent for this purpose has heretofore been unavailable. For example, Abciximab, marketed as ReoPro™ (manufactured by Centocor B. V., Leiden, The Netherlands, marketed by Eli Lilly and Co., Indianapolis, Ind.) is a 47,615 dalton Fab fragment of a chimeric human-murine monoclonal antibody designated as 7E3, that is purified from a cell culture supernatant. Abciximab (7E3) is stated to bind to the intact platelet GPIIb/IIIa receptor and inhibit platelet aggregation by preventing the binding of fibrinogen, von Willebrand factor, and other adhesive molecules to GPIIb/IIIa receptor sites on activated platelets. The mechanism of action is stated to involve steric hindrance and/or conformational effects to block access of large molecules to the receptor, rather than direct interaction with the RGD (arginine-glycine-aspartic acid) binding site of GPIIb/IIIa (ReoPro™ product label, also see, inter alia, U.S. Pat. Nos. 5,336,618, 5,275,812, 5,387,413, 5,770,198 5,440,020, 5,877,006, the disclosures of which are incorporated by reference herein). Abciximab is also reported to bind to the vitronectin (alphavbeta3 ) receptor found on platelets and vessel wall endothelial and smooth muscle cells (ReoPro™ product label).

Other anti-integrin antibodies, and related peptides binding such antibodies, are disclosed by, e.g., U.S. Pat. Nos. 5,196,511, 5,149,780, 5,262,520, the disclosures of which are incorporated by reference herein. These antibodies immunoreact with, e.g., an epitope formed by the RGD-binding region of an integrin beta subunit that is homologous to residues 110-170 of GPIIIa.

Despite these previous efforts, drugs currently utilized to counteract platelet function and/or thrombus formation in patients at risk for such disorders suffer from a lack of potency and/or specificity, and are often difficult to apply to a localized part of the circulatory system. While antibody-based agents such as Abciximab provide enhanced specificity to anti-platelet and/or thrombosis treatments, these antibodies are, at best, mouse-human hybrid proteins, and, despite the human components, the ReoPro™ product label warns that, “[a]dministration of Abciximab may result in human anti-chimeric antibody (HACA) formation that could potentially cause allergic or hypersensitivity reactions (including anaphylaxis), thrombocytopenia or diminished benefit upon readministration of Abciximab.”

In addition, production of Fab fragment agents such as Abciximab is a complicated and expensive process requiring expression in mammalian culture, followed by multiple process steps. These include proteolysis of the source mAb to generate the Fab fragment, followed by further processing and purification to obtain the commercial product. Thus, there remains a need in the art for an improved agent for inhibiting platelet aggregation that can be readily and more economically produced in quantity, e.g., by production in microbial culture systems.

Conjugating biologically-active proteins or enzymes to polymers has been suggested to improve one or more of the properties of circulating life, water solubility or antigenicity in vivo. For example, some of the initial concepts of coupling peptides or polypeptides to polyethylene glycol (PEG) and similar water-soluble polymers are disclosed in U.S. Pat. No. 4,179,337, the disclosure of which is incorporated herein by reference. Conjugates are formed by reacting a biologically active material with a several fold molar excess of a polymer which has been modified to contain a terminal linking group. Insulin and hemoglobin were among the first therapeutic agents conjugated. These relatively large polypeptides contain several free epsilon-amino attachment sites. Several polymers could be attached without significant loss of biologic activity.

Thus, there remains a need in the art for improved anti-platelet and/or anti-thrombosis agents having specificity available from antibody products, but with fewer of the difficulties associated with existing agents and drugs, and a particular need for such improved agents having the advantages provided by conjugation with substantially non-antigenic polymers.

### SUMMARY OF THE INVENTION

It is therefore an object of the invention to solve these and other problems in the art by providing novel human anti-platelet binding proteins for modulating and inhibiting platelet aggregation and/or thrombus formation by binding to platelet antigens in such a way as to inhibit platelet aggregation and/or thrombus formation. Preferably, the inventive anti-platelet binding proteins bind to, among other antigens, GPIIb/IIIa receptor sites in either activated and/or non-activated form. Broadly, the invention provides novel anti-platelet antibodies, antibody fragments and/or SCA® proteins.

Other aspects of the invention include conjugates of the provided novel antibodies and related proteins, with one or more substantially non-antigenic polymers.

Therefore, the invention provides for antigen-binding proteins or polypeptides, e.g., substantially isolated and purified antibodies, antibody fragments and/or SCA® proteins, that bind to at least one epitope required for platelet aggregation and/or thrombus formation. These epitopes include, for example, human platelet glycoprotein IIb (“GPIIb”), human platelet glycoprotein IIIa (“GPIIIa”), human platelet GPIIb/IIIa complex (in activated or non-activated form) human platelet alphaᵥbeta₃ vitronectin receptor(s), an amino-terminal sequence of leukocyte integrin alpha_{M} I domain ranging from about CyS₁₂₈ to about Ser₁₇₂, a peptide having the amino acid sequence of GCPQEDSDIAFLIDGSGSIIPHDF (SEQ ID NO:1) a corresponding peptide of GPIIb/IIIa having the sequence DYPVDIYYLMDLSYSMKDDLWSIQN (SEQ ID NO:2) and others known to the art, as well as combinations thereof.

Methods of eliciting, identifying and producing the inventive anti-platelet binding proteins are provided. Also provided are vectors and host cells. The invention thus provides, for example, a nucleic acid molecule, or its complement, that encodes an anti-platelet binding protein, wherein the nucleic acid is isolated from a phage display library by an in vitro selection process that comprises screening a diverse human antibody variable domain expression library against at least one human platelet antigen, e.g., as described supra, and the human antibody variable domain expression library expresses single-chain proteins. The human antibody variable domain expression library is optionally derived from human immune cell variable sequences and is screened by an in vitro selection process.

The anti-platelet binding proteins of the invention are thus identified by antigen screening of vector libraries expressing a diverse range of antigen-binding variants, for example, single-chain Fv (“sFv”) proteins with diverse binding specificities. The selected clones are expanded and produced in a suitable host system, either eukaryotic or prokaryotic. The most promising of these clones are then screened for inhibition of platelet aggregation and/or platelet mediated fibrin formation, under appropriate physiological conditions.

For example, the identified anti-platelet binding proteins have a Kd ranging from about 1 pM to 10 nM and k_{off} from about 10⁻⁵ sec⁻¹ to about 10⁻³ sec⁻¹. Further, the identified anti-platelet binding proteins have a molecular weight of about 24 kDa to about 30 kDa and multiples of these ranges when the proteins are in the form of multimers.

Conjugates of the inventive anti-platelet binding proteins with a substantially non-antigenic polymer or polymers, are also provided. In particular, while the inventive anti-platelet binding proteins of the invention are preferably derived from human sources, alternative embodiments contemplate non-human, e.g., murine sources for the variable domains and/or other regions of the binding protein. In the latter embodiments, conjugation to substantially non-antigenic polymers confers the additional benefit of a substantial reduction in the immunogenic properties of non-human epitopes. Preferably, the conjugates include anti-platelet binding proteins conjugated to polyalkylene oxide. More preferably, the polymer is polyethylene glycol, as specified in greater detail, infra. Such polymer conjugates provide beneficial improvements to the pharmacokinetics, stability, antigenicity and efficacy of the inventive anti-platelet binding proteins.

The invention further provides an expression vector that includes the nucleic acid operably linked to a promoter. Optionally, the promoter is inducible and/or tissue-specific in its expression. Tissue specificity can include, e.g., a tissue such as arterial endothelial cells, mammary gland cells, bladder epithelium, and combinations thereof. It is also contemplated that the promoter can be effective for expression in plant cells. Suitable promoters include, e.g., a beta-lactamase promoter, a lac promoter, a trp promoter, a phoA promoter, an araBAD promoter, a T7 promoter, derivatives of the lambda PL and PR promotor and an OL/PR hybrid promoter, to name but a few. The selected expression vector can be, for example, a plasmid, a phage, a cosmid, a retrovirus vector a DNA virus vector, a chromosomally integrated vector and/or any other art-known and suitable vector.

Host cells comprising or transformed by the above-described vector or vectors are also contemplated. Such host cells include, simply by way of illustration, mammalian cells transformed in vivo or ex vivo and used to produce the anti-platelet binding protein in culture and/or in vivo. Bacteria, such as E. coli, and yeast are also readily employed for protein production. It is also contemplated that the anti-platelet binding protein can be produced by a host organism, e.g., a non-human mammal comprising cells transformed by the above-described vector that produce the anti-platelet binding protein of the invention.

Also provided are methods of using anti-platelet binding proteins to inhibit platelet aggregation and/or platelet-mediated fibrin formation.

### DETAILED DESCRIPTION OF THE INVENTION

Accordingly, the present invention provides for novel anti-platelet and/or anti-thrombus binding protein or proteins optionally conjugated to one or more substantially non-antigenic polymers. For convenience of description, the novel anti-platelet and/or anti-thrombus binding protein or proteins are hereinafter described as anti-platelet binding proteins, although this is in no way intended to limit the description of the properties of the inventive binding proteins. Further, the use of singular terms for convenience in description is in no way intended to be so limiting. Thus, for example, reference to a composition comprising “an antibody” includes reference to one or more of such antibodies, and reference to “a polymer” includes reference to one or more of such polymers. It is also to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting, since the scope of the present invention will be limited only by the appended claims and equivalents thereof.

In order to more clearly appreciate the nature of the invention, the following definitions are provided. As used herein, the term, “antibody” refers to a protein consisting of one or more polypeptides substantially encoded by immunoglobulin genes or nucleic acid molecules that encode one or more fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An “antigen-binding site” or “binding site” refers to the part of an immunoglobulin molecule that participates in antigen-binding. The antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs.” Thus, the term FR refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding “surface.” This surface mediates recognition and binding of the target antigen. The three hypervariable regions of each of the heavy and light chains are referred to as “complementarity determining regions” or “CDRs” and are characterized, for example by Kabat et al., 1987 “Sequences of proteins of immunological interest,” 4th ed. U.S. Dept. Health and Human Services, Public Health Services, Bethesda, Md.

It has been shown that the function of binding antigens can be performed by fragments of a whole antibody. Example binding fragments include, the Fab fragment, that consists of V_{L}, V_{H}, C_{L} and C_{H1} domains; (ii) the Fd fragment that consists of V_{H} and C_{H1} domains; (iii) the Fv fragment that consists of the V_{L} and V_{H} domains of a single arm of an antibody, (iv) the dAb fragment (Ward, E. S. et al., Nature 341, 544-546 (1989) which consists of a V_{H} domain; (v) isolated CDR regions; and (vi) F(ab′)₂ fragments, a bivalent fragment comprising two Fab fragments linked by a disulphide bridge at the hinge region.

Although the two domains of the Fv fragment are coded for by separate genes, it has proved possible to incorporate a synthetic linker that enables them to be made as a single protein chain (known as single-chain Fv (sFv) (see, for example, Bird, R. E. et al., 1988 Science 242:423-426, Huston, J. S. et al., 1988 Proc. Natl. Acad. Sci., U.S.A. 85:5879-5883) by recombinant methods. These sFv fragments were assembled from genes derived from hybridomas expressing mAbs that had been previously isolated.

The anti-platelet binding protein of the invention is contemplated to include any suitable macromolecular configuration, including, simply by way of example, IgG, IgM, IgD, IgE, IgA and combinations and complexes thereof Preferably, the antibody-based embodiments that would otherwise support complement fixation are modified to prevent this function, e.g., by cleavage and/or removal of the Fc portion or fragment. More preferably, the anti-platelet binding protein is provided in the form of monoclonal antibodies.

Optionally, the anti-platelet binding protein is provided in the form of one or more of an Fab fragment, an Fv fragment, a light chain fragment, a heavy chain fragment, and combinations thereof.

Even more preferably, the human anti-platelet binding protein is an single-chain antigen-binding protein. Such an SCA® protein includes at least two human variable domains operably connected by a peptide linker, and the variable domains are the same or different, and independently selected from the group consisting of a human light chain variable domain, a human heavy chain variable domain, and combinations thereof.

For example, in a particularly preferred embodiment, the SCA® is a single-chain Fv (also referred to hereinbelow as an “sFv” protein, and also art-known as an scFv protein), which is encoded by a nucleic acid expression sequence able to express a single polypeptide chain that combines at least two human variable domains operably connected by a peptide linker, so that the expressed protein retains a functional conformation allowing it to bind to desired antigen(s).

A number of methods for eliciting and identifying antigen-binding proteins, such as polyclonal antibodies and monoclonal antibodies (“mAbs”) of desired binding characteristics, are known to the art. The anti-platelet binding proteins in accordance with the invention can be monoclonal or polyclonal antibodies. Techniques for preparing monoclonal antibodies may be found in, e.g., Stites, et al. (eds.), 1988, (Basic and Clinical Immunology (4th ed.), Lange Medical Publications, Los Altos, Calif.), Harlow and Lane, 1988, (Antibodies: A Laboratory Manual CSH Press; Goding, 1986 (Monoclonal Antibodies: Principles and Practice (2d ed.), Academic Press, New York) and Kohler and Milstein, 1975 (Nature 256:495-497), all of which are incorporated by reference herein in their entireties.

In further embodiments, the inventive anti-platelet binding protein or proteins is selected to bind exclusively to platelet GPIlb/IIIa, in its activated or inactivated form. Further still, the inventive anti-platelet binding protein is optionally selected to bind to the activated or inactivated GPIIb/IIa and to exhibit cross-reactivity with other integrins. In yet a further embodiment, the inventive anti-platelet binding protein is optionally selected to bind to the same and/or an overlapping epitope on GPIIb/IIIa as does ReoPro™.

In yet a further still embodiment, the inventive anti-platelet binding protein or proteins are selected and engineered to be multivalent, with a plurality of binding sites, capable of binding to the same or different epitopes.

The source of the variable domains employed by the inventive binding protein(s) is preferably mammalian, including, for example, human and/or murine and/or combinations thereof.

I. Anti-platelet Binding Proteins Identification by Screening Hybridomas

The ReoPro™ antibody product, described supra, was identified using conventional hybridoma technology, as described, for example, by Coller et al., in U.S. Pat. No. 5,275,812, incorporated by reference herein. In brief, ReoPro™ is an Fab fragment of an mAb engineered from murine variable domains and human antibody constant regions. The source of the murine variable domains is a murine mAb, designated by Coller et al. Id. as 7E3, that is described as derived from hybridomas generated from murine lymphocytes from mice immunized with platelets. Positive hybridoma clones were identified by screening in microtiter wells containing platelet enriched plasma and a fibrin coated bead suspension. The screening endpoint was prevention of agglutination of the fibrin-coated beads when supernatant from clones positive for 7E3 anti-platelet antibody was added to the microtiter wells.

Thus, in one embodiment, the inventive platelet binding protein comprises variable domains derived from mAbs screened and identified from mammalian sources, e.g., including human and/or murine sources, as described by Coller et al., Id.

Optionally, once one or more potentially suitable hybridomas, expressing anti-platelet mAbs is obtained, e.g., from a murine source, cDNA is obtained from the hybridoma(s) and DNA expressing the gamma/kappa chain genes is isolated and used as the starting point for expression of a chimeric murine-human Fab molecule in either mammalian or bacterial host cell. See, for example, Better et al., 1988 Science 240:1041-1043. Thus, for example, light chains that are optionally kappa or lambda are paired with the gamma heavy chain.

Human platelet antigens that are readily employed for eliciting or screening for anti-platelet antibodies include, for instance, disrupted human platelet membranes, and the more specific antigens of interest as described supra, in the presence of adjuvants, as required.

Thus, in one embodiment of the inventive methods, human lymphocytes that are likely to be activated against platelet antigen, e.g., isolated from patients with anti-platelet autoimmune disease, are then employed to prepare hybridomas for screening, using the standard methods described, simply by way of example, by Coller et al., Id. supra. to identify hybridomas secreting antibodies that inhibit platelet aggregation in the above-described bead test, or any other art-known screening test. The positive hybridoma clones are employed to isolate human mAbs that avoid the potential difficulties that attend administration of murine or chimeric human/murine therapeutic proteins.

In another embodiment transgenic organisms, including, for example, transgenic mice expressing human immunoglobulins, are constructed and employed to isolate human mABs or other suitable human proteins having anti-platelet properties. These can be constructed, for example using any of several screening methods as described herein, and the transgenic organism constructed according to the method of, eg., Yang et al., 1999 Cancer Res 59: 1236; Jakobovis, 1998 Exp. Opin. Invest. Drugs. 7:607-614; Mendez et al., 1997 Nat. Genet. 15:146-156, all incorporated by reference herein.

Further, mAbs produced by positive hybridoma clones are employed as therapeutic agents, e.g., in the form of mAbs or mAb fragments, preferably with the Fc portion removed. Optionally, art-standard methods are employed to provide vectors that include cDNA engineered from mRNA extracted from positive hybridoma clones. These vectors are engineered to express any suitable anti-platelet binding protein, eg., human anti-platelet SCA® protein. In a further optional embodiment, the mAbs, mAb fragments or other derivative human anti-platelet SCA® protein, are conjugated with substantially non-antigenic polymers. Methods for engineering SCA® proteins from mRNA, as well as methods of conjugating human anti-platelet binding proteins to suitable non-antigenic polymers, are described in detail, infra.

II. Anti-platelet Human Binding Proteins Identified by Screening Expression Libraries

Given the need to screen 10⁶ or greater numbers of diverse binding domains when starting with lymphocytes donated by non-immunized and non-autoimmune humans, combinatorial screening methods are generally preferred over more conventional hybridoma screening systems. The mammalian immune system is capable of producing an enormous diversity of binding specificities. A review of this subject is provided, for instance, by Walter and Tomlinson, 1996, Antibody Engineering, Id. at Chapter 6, pp 119 et seq. Essentially, diverse repertoires of antibody genes are generated during mammalian development in B-lymphocyte populations by the combinatorial rearrangement of relatively small numbers of basic building blocks. These include the V_{H} and V_{L} variable domains described above, D or diversity domains and J_{H} or J_{L} joining segments for the heavy and light variable domains, respectively. The antigen-binding site comprises three loops from the heavy chain variable domain (H1, H2 and H3) and three from the light chain variable domain (L1, L2 and L3), that are supported by a conserved beta-sheet scaffold.

In humans, there are up to 51 functional VH, about 30 functional D and six functional J_{H} segments which have been mapped to Chromosome 14. For light chains, which may be either kappa or lambda, up to 40 functional Vₖₚₚₐ and five functional Jₖₐₚₚₐ segments are located on Chromosome 2 and an estimated 30 functional V_{lambda} and four functional J_{lambda} segments are located on chromosome 22.

The rearrangment of V, D, and J segments introduces two levels of diversity into the antigen-binding site of the antibody. First, there is combinatorial diversity, that is the choice of one of several V_{H}, D, and J_{H} segments for the heavy chain variable domain and one of several Vₖₐₚₚₐ or V_{L} and Jₖₐₚₚₐ or J_{lambda} segments for the light chain variable domain, couples with the association of different heavy and light chains. Secondly, a great deal of diversity can be introduced at the V-(D)-J joins due to imprecise joining, the use of different D segment reading frames and the addition of N and P nucleotides at the join. Rearranged V genes are further diversified by somatic mutation during an immune response.

While many of these potential V_{L}/V_{H} clones are eliminated during development of individual organisms, ie., potential human donors of lymphocytes, it remains accurate to state that the somatic cell genetic diversity inherent in the genes encoding the human immune system can provide a highly diverse range of cDNA molecules encoding human V_{L} and V_{H} regions. This broad range of somatic cell genetic diversity can be exploited by construction of large libraries of cDNA molecules encoding such genetically diverse V_{L}/V_{H} combinations in a manner allowing screening for binding of antigens of interest against a plurality of V_{L}/V_{H} vector expression products. If necessary, the natural diversity of cDNA obtained from donated lymphocytes can be further enhanced, by a number of art-known methods for randomly mutating nucleic acid coding sequences, either in vitro or in vivo.

In general, the more recently developed techniques utilize combinatorial libraries of vectors expressing a genetically diverse library or libraries of antibodies, eg., in phage or similar vectors, screened against desirable antigen targets. See, e.g., Pope et al., 1996 Antibody Engineering, A Practical Approach, Chapter 1, pp 1-39 (Eds McCafferty et al., IRL Press, at Oxford University Press) as well as U.S. Pat. No. 5,969,108, employing phagemid vectors, the disclosures of which are incorporated by reference herein in their entireties. While phage display technology is one optional method for screening such libraries, any suitable display technology is readily employed, e.g., cell display and ribosome display.

One preferred vector for constructing an expression library is bacteriophage fd. Fd is a filamentous, single-stranded DNA phage which infects male Escherichia coli cells. Adsorption to the host sex pilus is mediated by the gene 3 protein (g3p) displayed at the tip of the virion. The amino-terminal domains of the three to five g3p molecules on each virion form knob-like structures that are responsible for binding the phage to the F-pilus, and the C-terminal domain is anchored in the phage coat. Parmley, et al., 1988 Gene 73:305-18 demonstrated that peptides could be displayed on the surface of the fd phage by fusion to the N-terminus of g3p. Phage with binding activities could then be isolated from random peptide libraries after repeated rounds of growth and selection for phage with the desired binding characteristics.

The range of proteins that can be displayed by fusion to the N-terminus of g3p include folded proteins, such as functional single-chain Fv fragments, bivalent and bispecific dimeric Fv fragments, and Fab fragments. McCafferty, 1990 Nature 348:552-4, Holliger, et al., 1993 Proc. Natl Acad. Sci., USA, 90:6444-8 and Hoogenboom, et al, 1991 Nucl. Acids Res 19:4133-7, respectively. This has allowed the selection of phage clones from a mixed population according to the binding characteristics of expressed library proteins.

Phagemid vectors combine both a plasmid and fd phage origin of replication and allow 100-fold higher efficiencies of transformation, and screening of larger libraries, relative to phage vectors. The g3p gene, described supra, is preferably equipped with restriction sites suitable for insertion of DNA sequences, such as sFv-encoding sequences, and the g3p is under the control of an inducible promoter e.g., lac, induced by (isopropyl)-β-D-thiogalactopyranoside (“IPTG”). This modified g3p is also engineered to be compatible with PCR primers effective to amplify antibody variable regions, e.g., as used by Orlandi et al. 1989, Proc. Natl Acad. Sci., USA 86:3833-7. Superinfection with a helper phage results in the packaging of the recombinant gene 3/SCA® fusion protein on the surface of the phage, encoded by the fusion insert to the N-terminus of g3p. Optionally, other fd phagemid coat proteins may be employed for the same purpose, including, simply by way of example the g8 (or gVIII) as described by Ladner et al. in U.S. Pat. No. 5,403,484, incorporated by reference herein.

While the fd phage and its derivatives are widely employed, alternative vectors can be readily employed in the inventive methods. For example, functional expression of an antibody fragment as a gene 8 protein fusion in M13 by Kang, et al., 1991, Proc. Natl Acad. Sci., USA 88:4363-6 has also been described.

Preferably, the phage or phagemid clones demonstrating high affinity binding to antigens of interest according to the invention are subcloned and expressed in E. coli to produce anti- platelet and/or anti-thrombin binding proteins, employing methods such as those described by Pluckthun et al., Antibody Engineering, A Practical Approach, Id. at Chapter 10, pp203-252, the disclosure of which is incorporated by reference herein.

In another embodiment of the inventive methods, the mRNA of a previously prepared hybridoma cell line expressing a monoclonal antibody of interest can be cloned into the phage-display format. An aliquot of 1×10⁷ cells should give up to 100 μg of total RNA, 10 μg of this is used per cDNA preparation. For the Pharmacia QuickPrep' mRNA kit, Pharmacia recommend using 5×10⁶ cells per oligo (dT) column. After ethanol precipitation and resuspending in 20 μl, this should be adequate for four cDNA preparations.

Antigen-binding proteins, such as sFv proteins, can also be constructed from hybridoma cells and expressed in a suitable host system, including E. coli, as described by Filpula et al., Antibody Engineering, A Practical Approach, Id. at Chapter 11, pp 253-266, incorporated by reference herein. Antigen-binding proteins according to the invention can also be expressed in Pichia pastoris, as described by Ridder et al., 1995, Biotechnology 13:255-260, or even in mammalian cells, as described, for example, by Bebbington et al., Antibody Engineering. A Practical Approach, Id. at Chapter 12, pp. 269-288.

Once expressed in a suitable host system, the library is screened for affinity for platelet antigens by any suitable method, e.g., affinity chromatography, panning, and/or adsorption to platelets or recombinant cells expressing platelet antigens, or to beads coated with antigens of interest, in order to identify candidate clones. The clones that are identified as producing candidate human anti-platelet binding proteins are then further screened for efficacy in inhibiting platelet aggregation and/or thrombus formation, in vitro, as described, for example, by employing a platelet competition assay as described by U.S. Pat. No. 5,114,842, incorporated by reference herein, and/or in inhibiting platelet mediated fibrin formation. The most successful candidates are then tested in vivo, to determine their pharmacokinetics, and to confirm the dose-response kinetics of specific parameters, such as the prolongation of bleeding time, receptor blockade, and inhibition of platelet aggregation in response to agonist, as were determined essentially as described e.g., by Gold, et al., 1990 J. Clin. Invest. 86: 651-659 and U.S. Pat. Nos. 5,877,006, and 5,976,532, all of which are incorporated by reference herein.

A. Construction Of Antibody Gene Libraries from Non-Immunized Human Donors

Broadly, construction of a phage display library, such as an sFv phage display library, is conducted by first obtaining messenger RNA (“mRNA”). mRNA is extracted from lymphocytes (e.g., from peripheral blood or spleen) by standard methods, e.g., phenol/SDS extraction, e.g., as described by Lo et al., 1992 (Hum. Antibodies. Hybridomas 3:123), incorporated by reference herein. Optionally, the mRNA can be extracted in greater purity using a commercially available oligo (dT)-affinity purification kit, that includes an oligo (dT) affinity column. One suitable commercially available kit is, eg., QuickPrep™ (Amersham Pharmacia, Catalog No. 27-9254-01). The QuickPrep™ oligo (dT) affinity column will extract sufficient MRNA from 50 ml of blood to conduct four cDNA reactions.

Whatever the method for isolating human lymphocyte mRNA, the V_{L}- and V_{H}- encoding cDNA sequences, respectively, are separately amplified using standard art-known reverse transcription (“RT”) and PCR methods. In brief, the cDNA is prepared by priming on mRNA isolated from human lymphocytes with oligonucleotide primers complementary to the first constant region of each heavy and light chain, respectively, and then amplified by PCR or are amplified by art-standard RT-PCR methods, e.g., as described in U.S. Pat. Nos. 5,310,652, 5,322,770 and 5,618,703, the disclosures of which are incorporated by reference herein in their entireties.

Suitable primers for RT production of cDNA libraries are readily determined by the artisan. One art-known method employs primers selected from the published sequences of human antibody constant regions, as described, for example, by Kabat et al., 1991 (“Sequences of Proteins of Immunological Interest” 5th Ed, US Dept of Health and Human Services, Bethesda, Md.), incorporated by reference herein.

An alternative art-known method employs random hexamers for the first priming step, as described, for example, by Pope et al., 1996, Antibody Engineering, A Practical Approach, Id. at pp 14-15. Pope et al. describe a method for RT production of murine cDNA which is the template for PCR amplifications of variable heavy and light sequences using the primers shown in Table 1. For PCR amplification of human variable heavy and light genes, there are six VHBACK primers and four JHFOR primers employed as described in the method of these authors. For the lambda light chains PCR amplifications are conducted by similar protocols.

TABLE 1
Primary Murine PCR Primers (restriction sites underlined)

| | | |
|---|---|---|
| VH1FOR-2 | TGA GGA GAC GGT GAC CGT GGT CCC TTG GCC CC | (SEQ ID NO:3) |
| VH1BACK | AGG TSM ARC TGC AGS AGT CWGG | (SEQ ID NO:4) |
| MJK1FONX | CCG TTT GAT TTC CAG CTT GGT GCC | (SEQ ID NO:5) |
| MJK2FONX | CCG TTT TAT TTC CAG CTT GGT CCC | (SEQ ID NO:6) |
| MJK4FONX | CCG TTT TAT TTC CAA CTT TGT CCC | (SEQ ID NO:7) |
| MJK5FONX | CCG TTT CAG CTC CAGCTT GGT CCC | (SEQ ID NO:8) |
| VK2BACK | GAC ATT GAG CTC ACC CAG TCT CCA | (SEQ ID NO:9) |

Wherein,

M=A or C

R=A or G

S=G or C.

The primers of Table 1 are derived from framework 4 (FR4) and framework 1 (FR1) sequences. Framework 4 corresponds to the J region of the variable sequence.

Optimal binding results for screening purposes are obtained by allowing the expressed V_{L} and V_{H} polypeptides to associate as dimers into Fv moieties or fragments. While separately expressed V_{L} and V_{H} polypeptides will associate as dimers in culture, there is a tendency of noncovalently linked Fv dimers to dissociate, resulting in the possibility of selection of clones with greater dimer stability. Therefore, it is preferable that the V_{L} and V_{H} polypeptides are expressed as an sFv proteins because it is easier to produce a single gene and a single gene product. In addition it is desirable that the association/affinity between the two variable domains be strong to minimize separation. This is most effectively accomplished by creating expression vectors for sFv proteins that encode, in a single product, the V_{L} and V_{H} tethered together with a polypeptide linker segment that allows for V_{L} and V_{H} pairing to form a functional Fv binding site.

sFv expression vectors can be prepared by the methods described, for example, by co-owned U.S. Pat. Nos. 4,946,778 and 5,260,203, the disclosures of which are incorporated by reference herein in their entireties. In brief, an sFv expression vector is selected to express an SCA® protein comprising a first polypeptide that includes a region encoding a V_{L} domain of an antibody, a second polypeptide that includes a region encoding a V_{H} domain of an antibody; and at least one peptide linker between these two domains. The encoded linker is of a size and relative position in the expressed sFv, so that upon expression, the V_{L} and V_{H} domains are included in a single-chain polypeptide, able to form an Fv binding site from the V_{L} and V_{H} domains, that can be screened for binding activity with an antigen of interest.

In one preferred embodiment, the bases encoding the peptide linker are incorporated between the regions encoding the V_{L} and V_{H} domains by PCR, e.g., employing primers selected to express the linker as a 15 residue linker peptide of (Gly₄Ser)₃, as described in detail by Pope et al., 1996 Id. at pp 17-20. Of course, the artisan will appreciate that this is provided as an example, and that primers that assemble other linker configurations can be readily prepared. Examples of suitable primers for generally copying human cDNA encoding V_{L} and V_{H} domains are into a single linked sequence are art-known, as described and tabulated, simply by way of example, by Pope et al., 1996, Id. at pp 14-15, as shown in Table 2, below.

TABLE 2
PCR Primers for inter-domain linker insertion

| LINKFOR | TGG AGA CTG GGT GAGCTC AAT GTC | (SEQ ID NO:10) |
|---|---|---|
| LINKBACK | GGG ACC ACG GTC ACC GTC TCC TCA | (SEQ ID NO:11) |
| The primers shown in Table 2, above, are used in a PCR reaction in the presence of an | | |
| # additional linker segment. One example of the additional DNA linker segment is: | | |
| # 5′-GGCACCACGGTCACCGTCTCCTCAGGTGGAGGCGGTTCAGGCGGAGGTGG | | |
| #CTCTGGCGGTGGCGGATCGGACATCGAGCTCACTCAGTCTCCA-3′ | | |
| # (SEQ ID NO:12). | | |

This linker may also be used in a double stranded from where the complementary strand is likewise synthesized. Pope et al., Id. supra, outlines the basic strategy in their FIG. 3, at page 13, incorporated by reference herein.

In order to screen the sFv library against a panel of antigens, the library of nucleic acid molecules is expressed under conditions that allow antigens of interest to be contacted with a large number, e.g., 10⁶ or greater, different expressed sFv proteins. Preferably, for optimum expression of the diverse encoded sFv proteins, the library is incorporated into a phagemid vector, prepared as a chimera between a plasmid, e.g., a pUC-type plasmid and a phage vector, e.g., an fd phage, as described by Pope et al., 1996, Antibody Engineering, A Practical Approach. Id. at pp 2-5. Restriction sites for insertion of the library into gene 3 of a phagemid vector are engineered by PCR mutagenesis. The artisan will appreciate that any convenient restriction sites can be employed with suitable primers. Simply by way of example, the primers described by Pope et al., Antibody Engineering, A Practical Approach, Id. are listed in Table 3, below.

TABLE 3
PCR Murine Primers for addition of restriction sites

| | |
|---|---|
| VH1BACKSfi | GTC CTC GCA ACT GCG GCC CAG CCG GCC ATG GCC CAG GTS |
| | MAR CTG CAG SAG TCWGG (SEQ ID NO:13) |
| JK1NOT10 | GAG TCA TTC TGC GGC CGC CCG TTT GAT TTC CAG CTT GGTGCC |
| JK2NOT10 | GAG TCA TTC TGC GGC CGC CCG TTT TAT TTC CAG CTT GGTCCC |
| JK4NOT10 | GAG TCA TTC TGC GGC CGC CCG TTT TAT TTC CAA CTT TGTCCC |
| JK5NOT10 | GAG TCA TTC TGC GGC CGC CCG TTT CAGCTC CAG CTT GGTCCC |

(SEQ ID NO:14, SEQ ID NO:15, SEQ ID NO:16 and SEQ ID NO:17, respectively).

M=A or C

R=A or G

S=G or C

W=A or T

The phagemid pCANTAB-5 is described by Pope et al., 1996, Id. Antibody Engineering, A Practical Approach, and is constructed from the phagemid pUC119 and provides unique SfiI and NotI sites for insertion and cloning of antibody genes such as SCA® Fv, i.e., sFv, proteinsas SfiI/NotI fragments at the N-terminus of gene 3. Export of g3p fusions to the periplasm is directed using the synthetic leader sequence. Further derivatives of this vector are also described by Pope et al., 1996, Id.,

For example, pCANTAB-5E (Pharmacia, Cat. No. 27-9901-01) that has an E tag inserted at the C terminus of the soluble antibody has also been developed. This E tag can be detected in ELISA with the anti E mAb available commercially from Pharmacia (Cat. No. 27-9412-02). This E tag can also be used for purification by affinity chromatography. (See, for example, Pharmacia Recombinant Phage Antibody System purification module 17-1362-01). A similar vector, pCANTAB-5myc, has a c-myc tag with a sequence derived from the c-myc oncogene (Munro, et al., 1986. Cell 46:291-300), inserted at the C terminus, instead of the E-tag. The c-myc tag can be detected and purified with the anti-c-myc mAb 9E10. As described by Pope et al., Id., supra, at page 5, a derivative of this, pCANTAB-6 has an additional tag of six histidines that is useful for affinity-chromatography purification (see also, McCafferty et al., 1994 Appl. Biochem. Biotechnol., 47:157-73 and Munro et al., 1986 Cell 46: 291-300, incorporated by reference herein in their entireties). In addition, pCANTAB-5myc, pCANTAB-5E, and pCANTAB-6 contain an amber codon inserted at the start of the gene 3 segment so that the inserted protein g3p fusion can be secreted as a soluble fragment in a non-suppressing E. coli strain such as HB2151, without recloning the gene.

However, in a suppressor strain such as TG1 the suppression of the amber codon is incomplete. Therefore, intact g3p antibody fusion protein and sFv antibody are produced in the same cell with a suppressor strain. Induction of expression, e.g., with the inducer IPTG (when the vector has the appropriate inducible promoter) yields production of sFv antibody with no g3p fusion protein. This is employed to further test the binding specificity and affinity of selected clones. Alternatively, rescue with M13K07 helper phage will give both phage particles displaying functional antibody (which can be used for selection) and soluble antibody in the same culture.

B. Mutagenesis of Expression Library

In yet another method of identifying suitable vectors encoding human anti-platelet binding proteins, cDNA encoding V_{H} and V_{L} domains are mutagenized, e.g., by error-prone PCR amplification or CDR mutagenesis, eg., as described by Johnson et al. 1996 (Antibody Engineering, A Practical Approach, Id. at Chapter 2, page 42 et seq.) or Sheets et al., 1998, Id. supra, the disclosures of which are incorporated by reference herein. The resulting mutagenized nucleic acids encoding V_{H} and V_{L} domains are then prepared as a phage or phagemid screening library, as described supra, and methods, as described in the next section, are conducted to identify binding proteins for the desired antigens of interest.

In order to enhance variability, shuffled phage sFv binding libraries are optionally constructed, as described, for example, by Schier et al., 1996, J. Mol. Biol. 255:28-43.

C. Screening of Expression Library

Each expression library of diverse antigen-binding proteins, e.g., sFv proteins, is screened for affinity to platelet and other thrombus-related antigens by any suitable art-known method, including, affinity chromatography, panning, or adsorption to platelets or host cells expressing antigens of interest. Elution from affinity matrices has been achieved by specific elution using the antigen (or a related compound) or non-specific elution using, for example, 100 mM triethylamine. Washing procedures remove non-specifically bound phage. The phage binds to and is eluted from the surface according to the affinity or the nature of the binding interaction. Eluted phage are then used to infect male E. coli cells expressing the F pilus, allowing recovery of phage encoding antibodies with the desired binding characteristics.

Panning of an sFv phage library for GPIIb/IIIa binders may be done with antigen immobilized on microtiter plate wells, or using streptavidin-magnetic beads/biotinylated antigen, or employing whole cells expressing the recombinant antigen. The general approach is described in the following protocol that is a minor modification of protocol 12 as described by Pope et al., Id., supra. Additional steps using increasing elution stringency may be employed if necessary as described by de Bruin R et al, 1999 Nat Biotechnol 17:397-399, incorporated by reference herein.

D. Antigens

Panning of an sFv phage library for GPIIb/IIIa binders requires a suitable antigen, e.g., a GPIIb/IIIa antigen, for candidate moieties to be screened against. The GPIIb/IIIa antigen may be a peptide ( SEQ ID NO:b 1 or SEQ ID NO:2, supra) or a purified GPIIb/IIIa protein or peptide motif or a recombinant cell line transfected with expressible genes for both subunits of the GPIIb/IIIa protein. Preferably, the antigen is human GPIIb, human GPIIIa, human GPIIb/IIIa complex (activated or nonactivated), human platelet alphaᵥbeta₃ vitronectin receptor(s), an amino-terminal sequence of leukocyte integrin alpha_{M} I domain ranging from about Cys₁₂₈ to about Ser₁₇₂, a peptide having the amino acid sequence of GCPQEDSDIAFLIDGSGSIIPHDF (SEQ ID NO:1) and combinations thereof Other art-known GPIIb/IIIa epitopes are optionally employed to screen for anti-platelet binding proteins. These can be used separately or in combination with the above-mentioned epitopes, and include GPIIb/IIIa epitopes and motifs believed to be functional GIIb/IIIa components, as described by Calvete, 1999, P.S.E.B.M. 222:29-38, incorporated by reference herein.

In one preferred embodiment, a GPIIb/IIa protein is purified from human platelets, e.g., by affinity chromatography, using any art-available GPIIb/IIIa binding moiety, such as an mAb or mAb fragment retaining GPIIb/IIIa binding properties. For example, mAb A₂A₉ (A29) (JS Bennett et al., 1983, Proc. Natl. Acad. Sci., USA, 80:2417-2421) and/or mAb SSA6 (Weisel, et al., 1992 J. Biol. Chem. 267(23):16637-16643) are readily employed for affinity chromatography of GPIIb/IIIa protein from human platelets, as exemplified hereinbelow. Alternatively, recombinantly produced GPIIb and/or IIIa or the full complex is optionally produced in a recombinant host and purified by standard methods, including, e.g., affinity chromatography as described above.

E. Assay of Candidate Clones

Purified sFv binding proteins produced by above-identified clones are further tested for competition with an antibody known to bind at high affinity to one or more platelet antigens of interest and for the ability to block platelet aggregation.

1. Competitive Binding Inhibition

Antibody competition assays are known, and any suitable method is readily employed for this screening. Simply by way of example, the methods of Harlow and Lane, 1988, Antibodies: A Laboratory Manual, Chapter 5, pp 567-573 or the biosensor or BIAcore method as described, for example, by Schier et al., J. Mol. Biol. 255:28-43 (both incorporated by reference herein) can be employed. Essentially, the assay determines whether a standard radio-labeled antibody is displaced by a candidate binder, from sites on GPIIb, GPIIIa, activated or non-activated GIIb/GPIIIa complex, and/or other platelet antigens of interest. Broadly, purified binding proteins produced e.g., from any of the above-described expression systems, are used to compete with radioiodinated competitive antibody, eg., ReoPro™, mAb A₂A₉ of Bennett et al., 1983, Id. as described supra, or other art-known antibodies for binding to human platelets. For instance, the method of Gold et al., U.S. Pat. No. 5,275,812, incorporated by reference herein can be applied, as follows.

Platelet-rich plasma (PRP) is prepared by centrifugation of citrated whole human blood at about 1900 rpm for about 2-5 minutes. ¹²⁵I-labeled competitive antibody (about 150,000 cpm) is added to the appropriate dilution of the purified sFv binding protein of interest, and the reaction initiated by adding 150 μl PRP. The assay is incubated for 1-2 hours at room temperature, and then platelets with bound antibody are separated from free ¹²⁵I-labeled competitive antibody by centrifugation, preferably through 30% sucrose at 12,000 g for about 3 to about 5 minutes in a microfuge tube, e.g., 0.4 ml. The resulting pellets are removed from the microfuge tubes, e.g., by cutting the pellet-containing plastic bottom tips off the microfuge tubes. The pellet-bound ¹²⁵I- is then measured by a gamma counter.

The competition for binding to platelets between ¹²⁵I-labeled competitive antibody and the same antibody without label is determined as a control or baseline. These controlled competition results are then compared to the competition between the ¹²⁵I-labeled competitive antibody and each unlabeled sFv binding protein of interest, to obtain ratios of competitive activity between competitive antibodies and candidate sFv binding proteins.

2. Inhibition of Platelet Aggregation

Purified sFv binding proteins produced by candidate clones are added to citrated whole human blood and incubated at 37 degrees C. for 10 minutes. The rate of platelet aggregation is measured after activation with collagen or ADP using a whole blood aggregometer (Chronolog Corp.).

3. ELISA Analysis of Screened Binding Proteins

Phage-based anti-platelet clones can be assayed directly for the ability to bind specific antigens of interest by immunoassay techniques such as ELISA. This is accomplished, for example, using the methods described by Pope et al., 1996 Id. For most antigens of interest, phage antibodies can be detected with antiserum raised against bacteriophage fd, in a suitable mammal, e.g., sheep. Commercial anti-fd antibody is available, e.g., from Pharmacia (Cat. No. 27-9402-01) and can be used to detect most antigens by ELISA assays.

In addition, soluble antibody preparations can be used in the ELISA and detected with a monoclonal antibody such as the antibody to the E tag (Pharmacia, cat. no. 27-9412-01) which will bind to the E tag present on antibody clones grown in the vector pCANTAB-5E. Similarly, solubly expressed antibody from clones in the vector pCANTAB-5myc or pCANTAB-6 can be detected with the monoclonal antibody 9E10 which will bind to the c-myc tag.

F. Production of Human Anti-Platelet Binding Protein(s)

1. Growth and Soluble Expression

Preferably, the phagemid clones selected as expressing a human anti-platelet binding protein, e.g., as an sFv protein, are engineered, as described supra, to express the sFv in soluble form. The phagemid, or other suitable vector to which the coding sequence is optionally transferred, is cultured and the produced soluble binding protein is isolated and purified by appropriate, art-known methods.

Simply by way of example, the methods of Pope et al., Id., are readily employed. The pCANTAB-5E. pCANTAB-5myc, pCANTAB-6 vectors (but not pCANTAB-5) have an amber codon between the Fv antibody sequence and gene 3, as mentioned, supra. Growth in a non-suppressor strain such as HB2151, in which the amber codon TAG is fully recognized as a stop codon, will allow soluble expression of, e.g., an sFv protein, or other analogous binder and/or antibody.

After the addition of an inducer appropriate for the promoter employed to construct the vector, e.g., IPTG [(isopropyl)-β-D-thiogalactopyranoside] the expression of sFv is induced. Soluble sFv is transported to the periplasm of the cell. From there it can leak or be excreted into the growth media. Soluble sFv can then be harvested either from the growth culture or from the periplasmic space. This latter is useful for large-scale production since it reduces the volume of crude extract prior to affinity column purification.

In an alternative embodiment, the identified clones are expressed and produced in E. coli by the methods of Filpula et al., 1996, Antibody Engineering, A Practical Approach, Chapter 11 pp 253-256, Id., and/or in mammalian cells by the methods of Bebbington, 1996, Antibody Engineering, A Practical Approach, Chapter 12, pp 269-288, and/or in Pichia pastoris, as described by Ridder et al., 1995, Biotechnology 13:255-260, and/or in Better et al., 1988, Id. supra, the disclosures of which are incorporated by reference herein.

2. Purification of Expressed Soluble Human Anti-Platelet Binding Protein

Several methods are available for purifying soluble antigens and/or antigen-binding proteins. For example, in one embodiment the expression vector is designed to provide for a polyhistidine tag. This allows the expressed protein to be purified by metal chelate affinity purification, e.g., by the method of McCafferty, et al., 1994 Appl. Biochem. Biotechnol. 47:157-73. Briefly, a metal chelate affinity column or analogous apparatus is prepared with Ni-NTA (nitrilo-tri-acetic acid) agarose (available from Qiagen) and is used according to the manufacturer's recommendations (e.g., in the elution buffer it is important to readjust the pH of PBS or saline when imidazole is added).

Alternatively, purification is conducted by affinity chromatography wherein the antigen of interest, e.g., a GPIIb, GPIIIa or other, is cross-linked to a Sepharose column (e.g., CNBr-Sepharose). In the case of small haptens, such as the peptide fragment antigens described herein, these can be cross-linked to a BSA-Sepharose column. Elution of bound antibody with 250 mM glycine buffer, pH 2.5, followed by neutralization and dialysis generally preferred. Binding proteins or antibody prepared in this way are normally stable when frozen at −20° C.

An additional purification method is described by Filpula et al., 1996, Antibody Engineering, A Practical Approach, Chapter 11, pp 264-265, wherein SCA proteins are purified by ion exchange chromatography and size exclusion chromatography.

III. Anti-platelet Human Binding Proteins Identified from Autoimmune Thromocytopenia Patients

There are reports in the clinical literature of patients with autoimmune thrombocytopenia. This is a severe disease of unknown etiology, characterized by phagocytic platelet destruction at rates that cannot be compensated for by increased production of platelets. Anti-platelet autoantibodies that react with intact GPIIb/IIIa have been isolated in such patients. For example, see Berchtold et al., 1998 Eur J Haematol 61(4):223-8, and Escher, et al., 1998 Br J Haematol 102(3):820-8, Jendreyko et al., 1998 Euo. J. Immunol. 28:4236-4247, all of which are incorporated by reference herein in their entireties.

Therefore, in yet another embodiment of the methods of the invention, serum donated by human patients with autoimmune thrombocytopenic purpura can be readily screened for antibodies having anti-GPIIb/IIIa binding activity, and binding to other platelet antigens of interest. cDNA encoding autoantibodies that prevent platelet aggregation are then employed as templates, by art-known methods, to prepare human anti-platelet binding proteins, and preferably, human SCA® anti-platelet binding proteins, employing the methods described infra.

Donors are selected from patients with a confirmed clinical diagnosis of thrombocytopenia, who are then screened to determine the presence of autoimmune anti-platelet antibodies in serum. The serum samples are tested for binding to platelets, and then more specifically are tested for binding to GPIIb/IIIa and/or other integrin-related epitopes as discussed supra. In addition, donors can be selected by screening clinical samples against intravenous immunoglobulin preparations, as described by Jendreyko, et al., 1998 Id., supra. The autoantibodies are then fractionated, and, for example, the Fv heavy and light chain variable domains sequenced, and DNA encoding these domains incorporated into vectors expressing sFv proteins.

In a more preferred embodiment, mRNA from lymphocytes obtained from donors positive for autoantibodies binding to GPIIb/IIIa, and/or other integrin-related epitopes is then isolated, amplified by reverse transcription to produce cDNA, which is then incorporated into vectors expressing sFv proteins in a combinatorial phage display system as described supra. Clones expressing sFv proteins positive for binding to desired platelet antigens are then expanded in suitable bacterial vectors, as previously described, and then are produced in quantity for further screening and production, also as described above.

IV. Conjugates with Non-antigenic Polymers

The invention further provides the anti-platelet binding proteins, e.g., the sFv proteins described supra, covalently attached or conjugated to substantially non-antigenic polymers. Useful polymers include, simply by way of example, poly(alkylene oxide)s (“PAOs”) and more preferably, poly(ethylene glycol)s (“PEGs”). Advantageously, such conjugates have reduced immunogenicity and antigenicity, as well as having a longer half-life in the bloodstream as compared to the parent protein. Monovalent and polyvalent anti-platelet binding proteins-polymer conjugates are encompassed by the invention.

In order to form the conjugates of the invention, polymers such as PAOs are converted into activated forms, as that term is known to those of ordinary skill in the art. Thus, one or both of the terminal polymer hydroxyl end-groups, (i.e. the alpha and omega terminal hydroxyl groups) are converted into reactive functional groups that allow covalent conjugation. This process is frequently referred to as “activation” and the product is called an “activated poly(alkylene oxide)”. Polymers containing both alpha and omega linking groups are referred to as bis-activated polyalkylene oxides. Other substantially non-antigenic polymers are similarly “activated” or functionalized.

Suitable polymers will vary substantially by weight, however polymers having molecular weights ranging from about 200 to about 60,000 are usually selected for the purposes of the present invention. Molecular weights of from about 5,000 to about 40,000 are preferred and 5,000 to about 20,000 are particularly preferred.

The polymeric substances included are also preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

As an alternative to PAO-based polymers, effectively non-antigenic materials such as dextran, polyvinyl pyrrolidones, polyacrylamides, polyvinyl alcohols, carbohydrate-based polymers and the like can be used. Indeed, the activation of alpha and omega terminal groups of these polymeric substances can be effected in fashions similar to that used to convert polyalkylene oxides and thus will be apparent to those of ordinary skill. Those of ordinary skill in the art will realize that the foregoing list is merely illustrative and that all polymer materials having the qualities described herein are contemplated. For purposes of the present invention, “effectively non-antigenic” means all materials understood in the art as being nontoxic and not eliciting an appreciable immunogenic response in mammals.

Among the substantially non-antigenic polymers, mono-activated polyalkylene oxides (PAO's), such as monomethoxy-polyethylene glycols are preferred. In alternative embodiments, homobifunctional bis-activated polymers such as bis-succinimidyl carbonate activated PEG are preferred.

The activated polymers are thus suitable for reacting with anti-platelet binding proteins and forming polymer conjugates thereto, wherein attachment preferably occurs randomly, or in a site-directed manner, e.g., at either the amino terminal alpha and/or epsilon-amino groups of lysines or engineered cysteines present on the anti-platelet binding proteins.

Preferably, the conjugates are prepared according to the methods of L. S. Lee et al., 1999 (Bioconjugate Chem. 10: 973-981), incorporated herein by reference. L. S. Lee et al. have reported protocols for the random, but delimited, PEGylation of sFv primary amine or carboxyl groups. In a further preferred method, conjugates of, e.g., SCA® and/or sFv proteins are prepared by linking to a polyalkalene oxide, such a PEG, by the site-specific conjugation methods described in co-owned international patent application WO98/48837, the disclosure of which is incorporated by reference herein. These protocols are applied to lead candidates of sFv specificities isolated by the above methods.

A. Selection and Design of Conjugation Sites

In those embodiments of the inventive conjugates for which particular conjugation sites are engineered, the binding of activated polymers is directed to portions of the anti-platelet binding proteins distal to the binding domain(s), and linkage is preferably to the constant regions of the anti-platelet binding protein and, in the case of SCA® anti-platelet binding proteins, the C-terminus and variable domain linker (between the V_{L} and V_{H} domains) are preferred as linkage sites.

In the present invention, conjugation sites, e.g., PEGylation sites, can be identified in a number of different regions, depending upon the particular structure of the identified SCA® binding proteins. Therefore, the design of polymer conjugation sites on an SCA® protein involves examining the structural information known about the protein, and the residues in the proteins involved in antigen-binding. The conjugation sites are chosen to be as far from these residues as possible so as to prevent disruption of the antigen-binding site. For example, useful Cys binding sites are typically located in the V_{L} and V_{H} regions distal from the antigen-binding site, the N-terminus of the polypeptide (V_{L}, V_{H} or neighboring site thereof), the linker region between the first and second polypeptide regions, or occur in a combination of these regions. In addition, oligo-Lys PEGylation sites can be located in the polypeptide linker or in the C-terminus or adjacent to the C-terminus of the polypeptide.

A useful Cys or oligo-Lys conjugation site can be located in (1) the native C-terminus of V_{L} (or V_{H}), (2) the C-terminus of V_{L} (or V_{H}) wherein the C-terminus has a deletion of one or plurality of amino acid residue(s), such that the remaining N-terminus amino acid residues of the peptide are sufficient for the conjugated binding protein to be capable of binding an antigen or (3) the C-terminus of V_{L} (or V_{H}) wherein the C-terminus has an addition of one or plurality of amino acid residue(s), such that the remaining N-terminus amino acid residues of the binding protein are sufficient for the conjugated product to remain capable of binding an antigen. By “native” is intended the naturally occurring C-terminus of the immunoglobulin variable sequence (Kabat et al., 1991 Id. supra). By “C-terminus” it is well understood in the art as intending the C-terminal amino acid residue or the C-terminal region of the polypeptide, which could include up to all of the amino acid residues of the polypeptide excluding the first N-terminal amino acid residue of the polypeptide. However, in the present invention, “C-terminus” is intended as the C-terminal amino acid residue of the above mentioned three types of C-terminus (1, 2, or 3), unless otherwise indicated or intended.

Simply by way of example, residues suitable for conjugation are identified and engineered within loop sites in regions of the SCA® that are diametrically opposed to the SCA® antigen-binding site(s). The five loop regions and C-terminal extension chosen as preferred sites of conjugation are preferably among the most distant regions spatially removed from the binding site. The engineered sites for site-specific conjugation may place engineered reactive moieties in the preferred sites. These groups may include (1) cysteines, (2) N-glycans, or (3) oligo-lysine segments.

As an example, the six furthest portions of an sFv from the antigen-binding site are:

1) The loop made up of residues 11 to 15 in the light chain;

2) The loop made up of residues 77 to 79 in the light chain;

3) The N-terminus of the linker;

4) The loop made up of residues 11 to 15 in the heavy chain;

5) The loop made up of residues 82B, 82C and 83 in the heavy chain; and

6) The C-terminus of the Fv (or sFv).

The residues are identified as according to Kabat et al., 1991 Sequences of Proteins of Immunological Interest, 5th ed., U.S. Dept. Health and Human Services, Bethesda, Md., incorporated by reference herein. These potential conjugation sites are determined by examining the 4-4-20 mouse Fab structure (see, Whitlow, M. et al., 1995, Protein Engng. 8:749-761, incorporated herein by reference in its entirety). These identified murine residues are also appropriate for human Fv engineering due to the considerable framework conservation between mouse and human.

After identifying the loops furthest from the antigen-binding site, the nucleic and amino acid sequences of each loop are examined for possible Cys PEGylation sites that may be engineered into the loop region. The engineered placement of the Cys residue anywhere in these six identified regions can generate a preferred site for sFv conjugation. The engineered placement of the oligo-Lys residues in the linker, the C-terminus of the sFv (or SCA®) and/or adjacent to the C-terminus of the sFv, can generate a preferred site for sFv conjugation to a polymer.

The particular nucleotide sequence which is used to introduce, e.g., a Cys or oligo-Lys conjugation site into the various positions will depend upon the naturally-occurring nucleotide sequence. The most preferred sites are those in which it takes a minimum number of changes to generate the PEGylation site. Of course, based on the redundancy of the genetic code, a particular amino acid may be encoded by multiple nucleotide sequences.

When required, site-directed mutagenesis is used to change the native protein sequence to one that incorporates the Cys residue or oligo-Lys residues for PEGylation. The mutant protein gene is placed in an expression system, such as bacterial cells, yeast or other fungal cells, insect cells or mammalian cells. The mutant protein can be purified by standard purification methods.

Oligonucleotide-directed mutagenesis methods for generating the Cys or oligo-Lys conjugation sites and related techniques for mutagenesis of cloned DNA are well known in the art. See, Sambrook et al., MOLECULAR CLONING: A LABORATORY MANUAL, 2nd ed., Cold Spring Harbor Laboratory, Cold Spring Harbor, N.Y. (1989); Ausubel et al (eds.), CURRENT PROTOCOLS IN MOLECULAR BIOLOGY, John Wiley and Sons (1987), both incorporated herein by reference herein. A preferred oligonucleotide-directed mutagenesis method for the present invention is according to Ho et al., 1989, Gene 77:51-59, incorporated herein by reference.

B. Polymers and Conjugation Methods

As noted supra, the conjugated polymers are preferably PAOs, and more preferably, polyalkylene glycols (“PAGs”), which term is intended to include both compounds wherein R¹ is hydrogen and compounds wherein R¹ is alkyl. “PEG” refers to poly(ethylene glycol) and “mPEG” refers to methoxy poly(ethylene glycol). In one preferred embodiment, the PAGs employed in the invention are straight chain polymers of the structural formula:

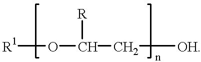

In another preferred embodiment, the PAG are branched chain molecules, as described, for instance, by published international patent application WO 98/48837 (pub. 1998), the disclosure of which is incorporated herein.

A preferred polyalkylene glycol for use in the present invention is poly(ethylene glycol)-hydrazine. The most preferred polyalkylene glycol for use in the present invention is methoxy poly(ethylene glycol). The PAG does not have to be of a particular molecular weight, but it is preferred that the molecular weight be between about 500 and about 40,000; more preferably, between about 5,000 and about 20,000. The choice of molecular weight of PAG is made based on the nature of the particular polypeptide employed, for example, the number of amino or other groups available on the polypeptide for modification. Molecular weights of about 10,000 and about 20,000 are most preferred.

It is well known in the art that PAGs that contain two terminal hydroxyl groups per moiety are capable of crosslinking other polymers, e.g., proteins. Where, as is often the case, crosslinking would be deemed undesirable, such crosslinking can be minimized or prevented by means known in the art. For example, Davis et al., in U.S. Pat. No. 4,179,337 have pointed out that a preferred means for preventing crosslinking is to preblock one end of the PAG, such as is done in the commercially available methoxy poly(ethylene glycol).

The PAGs employed in the practice of the present invention are preferably coupled to polypeptides by means of suitable coupling agents. A useful review of a number of coupling agents that can be employed in the practice of the present invention appears in Dreborg et al., 1990, Critical Reviews in Therapeutic Drug Carrier Systems 6(4):315-365, see, especially, pp. 317-320, as well as by WO 98/48837, Id. supra.

Probably the best known coupling agent for this purpose is cyanuric chloride. Its use has been described in numerous references, see, for example, Abuchowski et al., 1977, J. Biol. Chem. 252(11):3578-3581. Zalipsky et al., 1983 Eur. Pol. J. 19(12):1177-1183, among others, have described the reaction of methoxy poly(ethylene glycol) with succinic anhydride.

It is also known to alkylate MPEG with ethylbromoacetate in the presence of a base such as K-tertiary butoxide in tertiary butanol, Na-naphthalene in tetrahydrofuran, or butyl lithium in benzene. The terminal hydroxyl groups of PEG can be transformed into amine, carboxyl, or hexamethyl isocyanate groups. See, for example, Zalipsky et al., 1983, Id. supra. A mixed anhydride derivative of carboxylated mPEG can be prepared in the presence of triethylamine and then reacted with proteins.

These reactions produce activated polymers, e.g., activated PAGs. In one preferred embodiment, the activated PAG precursor includes succinimidyl carbonate (“SC”) derivatives (Zalipsky et al., 1995 Bioconjugate Chem. 6:133-151, incorporated by reference herein), e.g., SC-PEGs ranging in size from about 12,000 to about 40,000. Most preferred are SC-PEGs at the 12,000 and/or 20,000 size range. Additional preferred activated PEGs include thiazolidine-2-thione derivatives or T-PEGs (Greenwald et al., 1996 Bioconjugate Chem. 7:638-641, incorporated by reference herein) ranging in size from about 12,000 through about 40,000, or greater.

Conjugation reactions, sometimes referred to as PEGylation reactions, are generally carried out in solution with from about an equimolar to about a several fold molar excess of activated polymer. For example, the molar ratio of activated polymer to protein can range from about 1 to about 100 or greater. The process of the present invention, however, provides anti-platelet binding protein conjugates having high levels of retained activity by using anti-platelet binding protein engineered to have lysines on the inter binding domain linker and or in the constant domains, and by avoiding the use of an excessively high molar excess.

Thus, in a preferred embodiment that minimizes conjugation that blocks antigen-binding, the molar ratio of activated polymer to protein can range from about 1 to about 10, or more. More preferably, the molar ratio of activated polymer to protein ranges from about 1 to about 5, but more typically the molar ratio of activated polymer to protein is about 1 to about 2. In most circumstances the latter ratio will substantially avoid conjugating residues associated with the active high affinity binding site.

The conjugation reaction is also carried out under relatively mild conditions to avoid inactivating the anti-platelet binding proteins. Mild conditions include maintaining the pH of the reaction solution in the range of 6-8 and the reaction temperatures within the range of from about 0-30° C. and preferably at about 4° C. for about one hour. Suitable buffers include buffer solutions able to maintain the preferred pH range of 6-8 without interfering with the conjugation reaction. A non-limiting list of suitable buffers includes, e.g., phosphate buffer, citrate buffer, acetate buffer.

Although the reaction conditions described herein may result in some unmodified anti-platelet binding proteins, the unmodified anti-platelet binding proteins can be readily recovered and recycled into future batches for additional conjugation reactions.

In one embodiment, the conjugation reactions of the present invention initially provide, for example, a reaction mixture or pool containing anti-platelet binding protein conjugates having from about 1 to about 5 strands of polymer per anti-platelet binding protein. After the unreacted species have been removed, compositions containing the anti-platelet binding protein-polymer conjugates are recovered. These compositions retain the biological activity associated with the native or starting anti-platelet binding proteins, as measured using any art-standard assay, as discussed in more detail infra. Generally, the conjugates retain from about 1% to about 100% of the biological activity of the non-conjugated anti-platelet binding protein. Preferably, at least about 5% of the biological activity is retained, more preferably, at least about 10%, and even more preferably, at least about 50% of the biological activity is retained. Accordingly, the ranges of retained biological activity of conjugates according to the invention are from about 5% to about 99% of the biological activity, and more specifically, from about 30% to about 95% of the biological activity associated with non-conjugated anti-platelet binding proteins.

A representative conjugation reaction is set forth below.

An about 5-fold molar excess of activated polymer is dissolved in Water For Injection (pH approximately 6.0) and then added to an anti-platelet binding protein solution adjusted to about pH 8.0 with a suitable buffer such as a phosphate or borate buffer. The reaction is allowed to incubate at about 4° C., at about pH 8.0, for a suitable time, such as about 1 hour, with continuous gentle mixing. Thereafter, the conjugation reaction is stopped, for example with a several-fold molar excess of arginine or glycine. The unmodified anti-platelet binding protein present in the reaction pool, if any, after the conjugation reaction has been quenched, can be recovered for recycling into future reactions using ion exchange or size exclusion chromatography or similar separation techniques. Preferably, solutions containing the conjugates of the present invention contain less than about 5% unmodified anti-platelet binding protein.

If desired, the anti-platelet binding protein - polymer conjugates are isolated from the reaction mixture to remove high molecular weight species, and unmodified anti-platelet binding protein. The separation process is commenced by placing the mixed species in a buffer solution containing from about 1-10 mg/ml of the anti-platelet binding protein polymer conjugates. Suitable solutions have a pH of from about 6.0 to about 9.0 and preferably from about 7.5 to about 8.5. The solutions preferably contain one or more buffer salts selected from KCl, NaCl, K₂HP₄, KH₂PO₄, Na₂HPO₄, NaH₂PO₄, NaHCO₃, NaBO₄, and NaOH. Sodium phos buffers are preferred.

Depending upon the reaction buffer, the anti-platelet binding protein polymer conjugate solution may first have to undergo buffer exchange/ultrafiltration to remove any unreacted polymer. For example, the PAO-anti-platelet binding protein conjugate solution can be ultra-filtered across a low molecular weight cutoff (10,000 to 40,000 Dalton) membrane to remove most unwanted materials such as unreacted polymer, surfactants, if present, or the like.

Fractionation of the anti-platelet binding protein-polymer conjugates, if desired, can also be carried out using an anion exchange chromatography medium. Such media are capable of selectively binding PAO-anti-platelet binding protein conjugates via differences in charge which vary in a somewhat predictable fashion. For example, the surface charges of anti-platelet binding protein are determined by the number of available charged amino acids on the surface of the protein. Of these charged amino acids, lysine residues serve as the point of potential attachment of polyalkylene oxide conjugates. Therefore, conjugates of different variations of the anti-platelet binding proteins will have typically have a different charge from the other variations, to allow selective isolation.

In one preferred embodiment, strongly polar anion exchange resins, such as quaternary amine anion exchange resins are employed to isolate conjugates. Included among the commercially available quaternary anion exchange resins suitable for use with the present invention are Q-HD, QA TRISACRYL® and QMA_SPHEROSIL®, quaternary amine resins coated onto a polymer matrix, manufactured by IBF of Garenne, France, for Sepracor of Marlborough, Mass.; TMAE650M®, a tetramethylamino ethyl resin coated onto a polymer matrix, manufactured by EM_Separators of Gibbstown, N.J.; QAE550C®, and SUPERQC®, each a quaternary amine resin coated onto a polymer matrix and manufactured by TosoHaas of Montgomeryville, Pa. QMA Accell, manufactured by Millipore of Millford, Mass. and PEI resins manufactured by J T Baker of Phillipsburg, N.J., may also be used. Other suitable anion exchange resins e.g., DEAE resins can also be used.

For example, the anion exchange resin is preferably packed in a column and equilibrated by conventional means. A buffer having the same pH and osmolality as the polymer conjugated anti-platelet binding protein solution is used. The elution buffer preferably contains one or more salts selected from KCl, NaCl, K₂HPO₄, KH₂PO₄, Na₂HPO₄, NaH₂PO₄, NaHCO₃, NaBO₄ and (NH₄)2CO3. The conjugate-containing solution is then adsorbed onto the column with the high molecular weight species and unreacted polymer not being retained. At the completion of the loading, a gradient flow of an elution buffer with increasing salt concentrations is applied to the column to elute the desired fraction of polyalkylene oxide-conjugated anti-platelet binding proteins. The eluted pooled fractions are preferably limited to uniform mono- and bis-anti-platelet binding protein polymer conjugates after the anion exchange separation step. Any unconjugated anti-platelet binding protein species can then be back washed from the column by conventional techniques. If desired, the anti-platelet binding protein can also be separated via additional ion exchange chromatography or size exclusion chromatography. The temperature range for elution is between about 4° C. and about 25° C. Preferably, elution is carried out at a temperature of from about 6° C. to about 22° C. Fraction collection may be achieved through simple time elution profiles.

Since sFv protein typically have an isolectric point between 8.0 and 9.4, the conjugates of the invention are preferably purified by cation exchange chromatography, using art-known methods. This can be accomplished, e.g., using the “S” series of resins from Amersham Pharmacia, as described in the Examples, below.

Methods of Inhibiting Platelet Aggregation and/or Thrombus Formation

Another embodiment of the present invention provides methods of inhibiting platelet aggregation and/or thrombus formation, by application and/or administration of the inventive binding proteins, conjugates, and vectors or host cells expressing the inventive binding proteins, either alone or as adjuncts to administration of other art-known agents or methods. Some of the useful applications of the instant invention include, but are not limited to, inhibiting platelet aggregation and/or thrombus formation in the following situations: venous thromboembolism, unstable angina, saphenous vein bypass grafts, percutaneous transluminal coronary angioplasty, atrial fibrillation, valvular heart disease, cerebrovascular disease, peripheral vascular disease, secondary prevention of arterial thromboembolism, primary prevention of arterial thromboembolism, acute disseminated intravascular coagulation, chronic disseminated intravascular coagulation (Trousseau's syndrome).

In a further important application, the anti-platelet binding protein and/or autologous transformed or transfected host cells, are coated or bound to the surfaces of medical devices and/or appliances to prevent thrombus formation, prevent occlusion of extracorporeal devices, e.g., intravascular cannulas, prosthetic heart valves, vascular access shunts in hemodialysis patients, hemodialysis machines, and cardiopulmonary bypass machines, reducing the incidence of axillary-subclavian venous thrombosis in patients with long-term indwelling central vein catheters, and the like.

It will be appreciated, as mentioned supra, that the inventive anti-platelet protein, or vectors or cells expressing the inventive protein, can be co-administered or employed sequentially and/or in combination with other suitable anti-platelet, anticoagulant and anti-thrombus and/or thrombolytic agents. Such additional agents are described, for example, by Goodman & Gilman's The PHARMACEUTICAL BASIS OF THERAPEUTICS, Ninth Ed., Ed. Hardman et al. McGraw Hill, the disclosure of which is incorporated by reference. Such additional agents include oral anticoagulants such as derivatives of coumarin, including warfarin, dicumarol, phenprocoumon, acenocoumarol, and ethyl biscoumacetate, as well as indandione derivatives such as anisindione and phenindione. Other additional agents include, e.g., thrombolytic drugs for fibrinolysis and thrombolysis such as alpha2-antiplasmin, streptokinase, streptokinase-plasminogen complex (anistreplase; eminase), tissue plasminogen activator (T-PA), urokinase and aminocaproic acid, as well as anti-platelet drugs such as aspirin, dipyridamole and ticlopidine.

The advantages of the inventive anti-platelet protein are preferably enhanced by conjugation to substantially non-antigenic polymers, as discussed in detail, supra. In particular, the presence of one or more polymer molecules, e.g., polyethylene glycol (“PEG”) conjugated to the anti-platelet protein protects the protein from renal clearance and/or certain proteolytic processes. The result is a prolonged presence in tissue or blood, relative to the non-conjugated protein. Simply by way of illustration, the plasma concentration of systemically administered conjugated anti-platelet protein is maintained at an elevated level for longer time periods, relative to the non-conjugated protein. In particular, the area under the concentration curve or “AUC” for the conjugated protein ranges from about 2 to about 100 fold greater than that exhibited by the non-conjugated form of the inventive anti-platelet protein.

While not wishing to be bound by any theory or hypothesis as to the operation of the inventive conjugates, it is also believed that the conjugated anti-platelet protein will have the added advantage of providing steric protection and/or a hydration shell that will extend the reach, i.e., increase the area of blockade of the target platelet surface, per molecule of platelet bound conjugate. In addition, the conjugated polymer, which is substantially non-antigenic, will significantly reduce the likelihood of the patient forming an immune response to the conjugated anti-platelet binding protein. In particular, for each molecule of anti-platelet protein bound to a tissue to be protected, such as an arterial endothelial surface, an additional one, two or more conjugated molecules of polymer are also present adjacent to the binding site. These polymer molecules, having molecular weights ranging, eg., from about 5,000 to about 40,000 Daltons, are believed to provide steric protection of adjacent cell surface receptors and binding sites, thus greatly magnifying the effectiveness of each mole of conjugated anti-platelet protein.

A. Parenteral Administration of Anti-Platelet Binding Protein or Conjugated Binding Protein

The methods include administering an effective amount of anti-platelet binding protein, and polymer conjugates thereof, which have been prepared as described herein, to a mammal or to tissue of a mammal, including humans, in need of such treatment. The anti-platelet binding proteins, and polymer conjugates thereof, are useful for treating anti-platelet binding protein-susceptible conditions or conditions which would respond positively or favorably as these terms are known in the medical arts to anti-platelet binding protein-based therapy. Administration is typically by a parenteral route, given the protein nature of this therapeutic protein and its polymer conjugates, but is not exclusively limited to this route under conditions where oral, transdermal, nasal or pulmonary administration is appropriate.

Thus, without limitation, the anti-platelet binding proteins and conjugates can be used as a primary or adjunct agent to inhibit platelet aggregation and/or thrombus formation in patients undergoing medical or surgical procedures with a high risk of thrombus formation. For example, percutaneous interventions such as balloon angioplasty, atherectomy, stent placement, coronary bypass surgery, and the like. Optionally, the anti-platelet binding protein is administered to a patient in need thereof with concomitant heparin dose regimens. Unless contraindicated, aspirin by the oral route is also given prior to and during such procedures. The administration of anti-platelet binding protein or conjugate and any co-administered heparin, aspirin or the like, are also optionally continued during the recovery period while thrombus formation remains a potential threat.

The amount of the anti-platelet binding protein and/or anti-platelet binding protein polymer conjugate administered to treat or prevent the conditions described above is based on the platelet aggregation inhibitory properties and/or thrombus formation inhibitory properties of the inventive anti-platelet binding protein that is employed. It is an amount that is sufficient to significantly effect a positive clinical response. The maximal dose for mammals, including humans, is the highest dose that does not cause clinically important side effects. For purposes of the present invention, such clinically important side effects are those which would require cessation of therapy such as, for example, excessive bleeding, thrombocytopenia, hypersensitivity reactions and/or other immunogenic reactions and any other negative effect the risks of which outweigh the benefits of treatment.

Naturally, the dosages of the anti-platelet binding protein, in conjugated or unconjugated form, also varies somewhat depending upon the platelet inhibitory potency of the particular anti-platelet binding protein, e.g., dosages may be reduced substantially if the inventive binding protein is multivalent or has a higher binding affinity relative to other binding proteins that might be administered. For polymer conjugates, doses will also depend upon the substantially non-antogenic polymer selected, the molecular weight of the polymer, and the linker that is employed to form the conjugate, and the in vivo rate of hydrolysis of the linker.

In general, however, the non-conjugated anti-platelet binding protein, can be administered, for example, as an initial intravenous or intra-arterial bolus of about about 0.05 mg/kg to about 5 mg/kg. The initial bolus is optionally followed by a continuing infusion of the anti-platelet binding protein at a rate of about 1 to about 100 mg/min. More preferrably, the continuing infusion is conducted at a rate from about 10 μg/min to about 10 mg/min. The continuing infusion is maintained, for example, for a time period ranging from about 5 hours to about two weeks, or greater, depending upon the clinical requirements.

The polymer conjugated anti-platelet binding protein, can be administered, for example, as an initial intravenous or intra-arterial bolus ranging from about 0.1 mg/kg to about 1.0 mg/kg or more preferably from about 0.01 mg/kg to about 2 mg/kg. The initial bolus is optionally followed by a continuing infusion of the anti-platelet binding protein at a rate ranging from about 0.1 to about 50 mg/min, or more preferrably, ranging from about 0.001 mg/min to about 5 mg/min, for a time period ranging from about 5 hours to about two weeks, or greater, depending upon the clinical requirements. Of course, the ranges set forth above are merely illustrative, and those skilled in the art will determine the optimal dosing of the conjugate selected based on clinical experience and the treatment indication.

The anti-platelet binding protein-polymer conjugates of the present invention can be included in one or more suitable pharmaceutical compositions for administration to mammals. The pharmaceutical compositions may be in the form of a solution or suspension, or the like, suitable for intravenous or intra-arterial administration, and prepared according to methods well known in the art.

B. Administration of Anti-Platelet Binding Protein by Expression Vector

In a further embodiment of the invention, nucleotide molecules encoding anti-platelet binding protein according to the invention are employed to transfect or transform host cells that beneficially express and secret the desired anti-platelet binding protein, in situ.

Thus, the cells or tissues of patients in need thereof are transfected with vectors which introduce a gene expressing anti-platelet binding protein. This can be done either in vivo or in vitro. When conducted in vitro, the transfected cells or tissues are then optionally administered to a patient. Simply by way of example, a patient who has received balloon angioplasty is beneficially treated by administration of a suitable expression vector into the section of artery that undergoing angioplasty (either during or after the angioplasty procedure). The expression vector is engineered to produce the anti-platelet binding protein in a form that is secreted by the endothelial cells lining the artery, thus inhibiting thrombus formation in an artery section that has been injured. Of course, other anti-platelet, anticoagulant and/or anti-thrombosis agents can be co-administered, locally or systemically, to provide further protection against thrombosis in a high risk patient. Such additional agents are described, for example, by Goodman & Gilman's The PHARMACEUTICAL BASIS OF THERAPEUTICS, Ninth Ed. Ed. Hardman et al. McGraw Hill, the disclosure of which is incorporated by reference herein. In addition, vein segment(s) are typically employed as grafting material for arterial bypass surgery (e.g., typically coronary artery bypass, but not limited to that procedure). It is also contemplated that the internal lining of such harvested vein segments is contacted with vectors, or even “naked” or unpackaged nucleic acid segments suitable for transfection and expression, with secretion, of human anti-platelet binding protein to inhibit thrombus formation on, or adjacent to, the endothelial lining of the reimplanted vein segment.

Transfection and/or transformation of autologous host cells and/or tissues can be accomplished by any suitable art-standard methods, as described, for example, by Chen S-Y et al., 1997 Nature 385:78-80 (SCA®-toxin expressing conjugate inserted into autologous lymphocytes by a pCMV-23sFv-PEA40 vector); and Nicolet C M et al., 1995 Cancer Gene Therapy 2:161-170 (particle-mediated insertion of genetic material encoding an SCA protein by a “gene-gun”) the disclosures of which are incorporated by reference herein.

It will also be appreciated that the anti-platelet binding protein of the invention is readily adapted to be expressed as a fusion protein in combination with any protein or polypeptide-based fibrinolytic agent, e.g., a protease such as TP-A, to provide an agent with both thrombolytic and anti-thrombus formation properties. For example, Neblock D S et al., 1992, Bioconjugate Chemistry 3: 126-131, describes chemically cross-linking an anti-platelet murine mAb F(ab′)2 fragment (binds to GPIbI/IIIa) with a TPA activator, in order to provide an agent that both inhibits platelet aggregation and provides a localized TPA activator effect. However, expression of a fusion vector expressing the anti-platelet binding protein together with TP-A also provides localized fibrinolytic activity, with the added safety advantage provided by the human origin of the instant binding protein, plus the additional advantage, in certain preferred embodiments, as described supra, for in situ expression and secretion of the fusion protein, localized by autologous transfection or re-grafting to tissues at greatest risk of thrombus formation.

C. Other Utilities for the Anti-Platelet Binding Protein

The anti-platelet binding proteins according to the invention provide a wide range of utilities, in addition to the above-described therapeutic methods. These additional utilities include, simply by way of example, diagnostic imaging and assays, both in vivo and in vitro, purification and isolation of platelet antigens, and as receptors in electronic biosensors. The anti-platelet binding proteins are readily employed in immobilized form to purify and isolate platelet and related antigens, or in detectably labelled forms. The anti-platelet binding proteins can also be readily employed in combination with, either conjugated or non-conjugated, other therapeutic or diagnostic agents requiring selective localization or delivery to areas of platelet mediated thrombus formation in an animal, including a human patient.

The anti-platelet binding proteins can further be utilized as coatings, e.g., conjugated to polymer surfaces of laboratory instruments such as blood analysis equipment, clinical probes, such as catheters and the like, and machinery, such as heart-lung and dialysis machines and membranes thereof, to prevent undesired deposits of platelets and fibrin. These uses provide useful alternatives and/or supplements to heparin treatments and coatings, that do not always provide full and lasting protection from the formation of undesirable thrombus deposits onto such devices.

1. In Vitro Assays

The anti-platelet binding protein of the invention can readily be substituted for other binding proteins and/or antibody-based reagents in any art-standard assay or diagnostic tests. For example, the assay described by U.S. Pat. No. 5,246,832, incorporated by reference herein in its entirety, determines thrombin reactivity of platelets in a whole blood sample by adding to the whole blood sample a first antibody which is specific for an alpha-thrombin activated surface antigen of the platelets, then adding alpha-thrombin to the whole blood sample to activate a surface antigen of the platelets, wherein the first antibody binds to the activated surface antigen of the platelets, binding to the platelets a second antibody specific for another surface antigen of the platelets, and detecting the amount of the first and second antibodies bound to the platelets. In this assay, the first antibody is preferably an anti-GPIIb/IIIa binder, and the anti-platelet binding protein of the invention will readily be substituted.

2. In Vivo Diagnostics

Similarly, the anti-platelet binding protein of the invention is readily conjugated to any art-standard emitting isotope and/or radio-opaque label, and employed to localize thrombus formation in the heart or other body regions. Advantageously, the instant inventive binding proteins are human in origin, and thus less likely to provoke untoward reactions during repeated diagnostic administration. Also, in certain preferred embodiments, the instant inventive binding proteins are, for example, sFv proteins, which are significantly smaller than intact antibodies, and therefore able to penetrate and provide diagnostic information in otherwise restricted tissue spaces, particularly in tissue spaces with significant thrombosis activity.

The artisan will readily appreciate that the anti-platelet antigen-binding proteins are readily linked, by art-known methods, to any suitable label. Simply by way of example, these include radioisotopes such as 131Iodine, 125Iodine, 99mTechnetium and/or 111Indium by art-standard methods to produce radioimmunoscintigraphic agents. There are a number of art-known methods of linking a protein or peptide to Tc-99m, including, simply by way of example, those shown by U.S. Pat. Nos. 5,328,679, 5,888,474, 5,997,844, 5,997,845, incorporated by reference herein. Alternatively, a metal-binding domain can be engineered into the anti-platelet antigen-binding protein(s) to provide a site for labeling, e.g., a domain derived from a metallothionein protein.

In a further alternative embodiment, the polymer-conjugated anti-platelet antigen-binding proteins can be readily labeled by conjugation to a suitable polymer having one or more radioimmunoscintigraphic agents linked thereto.

It should also be mentioned that such imaging labels need not exclusively be radioimmunoscintigraphic agents, but can be any suitable agents for the required imaging situation, including, simply by way of example, radio-opaque (e.g., X-ray contrast), non-radioactive isotopic labels suitable for magnetic resonance imaging, fluoresence-type labels, labels exhibiting visible colors and/or capable of fluorescing under ultraviolet, infrared or electrochemical stimulation, to allow for imaging thrombi during surgical procedures, and so forth.

Broadly, for anatomical localization of a thrombus in a patient, the platelet-specific chimeric immunoglobulin is administered to a patient suspected of having a thrombus or thrombi. After sufficient time to allow the labeled immunoglobulin to localize at the thrombus site, the signal generated by the label is detected by a photoscanning device such as a gamma camera. The detected signal is then converted to an image of the thrombus. The image makes it possible to locate the thrombus in vivo and to devise an appropriate therapeutic strategy.

### EXAMPLES

Many modifications and variations of this invention can be made without departing from its spirit and scope, as will be apparent to those skilled in the art. The specific embodiments described below are offered by way of example only, and the invention is to be limited only by the terms of the appended claims, along with the full scope of equivalents to which such claims are entitled.

### Example 1

### Construction of sFv Phage Display Library

An sFv phage display library is constructed. The method of Sheets et al., 1998, Id. supra, is followed with minor modifications.

Total RNA is prepared from collections of human spleen cells and human peripheral blood lymphocytes. cDNA is synthesized from total RNA primed with a primer (e.g., 5′-TGG AAG AGG CAC GTT CTT TTC TTT-3′) (SEQ ID NO:18) which anneals to IgM specific variable gene mRNA. The V_{H} gene repertoires are amplified via PCR from this cDNA using Vent DNA polymerase (New England Biolabs) in combination with the above primer plus an equimolar mixture of primers. For example:

| | |
|---|---|
| 5′-CAG GTG CAG CTG GTG CAG TCT GG-3′ | (SEQ ID NO:19); |
| 5′-CAG GTC AAC TTA AGG GAG TCT GG-3′ | (SEQ ID NO:20); |
| 5′-GAG GTG CAG CTG GTG GAG TCT GG-3′ | (SEQ ID NO:21); |
| 5′-CAG GTG CAG CTG CAG GAG TCG GG-3′ | (SEQ ID NO:22); |
| 5′-GAG GTG CAG CTG TTG CAG TCT GC-3′ | (SEQ ID NO:23); and |
| 5′-GAG GTA CAG CTG CAG CAG TCA GG-3′ | (SEQ ID NO:24), |

that anneal to the N-terminal region of the V_{H} segment.

PCR products are purified from agarose gels and subjected to another PCR reaction wherein the primer sequences above have extensions with sequences corresponding to restriction sites (e.g. NotI and NcoI) useful for subsequent cloning. This PCR employs Tth DNA polymerase (Epicentre Technologies, Madison, Wis.) and an equimolar mixture of the two new modified primers.

The PCR products are digested with the relevant restriction enzymes (e.g. NcoI and NotI) and purified by agarose electrophoresis. These DNA fragments are ligated into a suitable plasmid (e.g plasmid pCITE4A, Novagen) and transformed by electroporation into a suitable E. coli host (e.g. TG1).

Using the products of about 5 to 15 ligation reactions and about 10 to 30 electroporations, a library of V_{H} genes containing 0.5×10⁸ to 1×10⁹ members is generated. Next the complete sFv library is generated by the following methods.

The V_{H} genes are amplified by PCR from the above V_{H} library using 100 ng-1 μg of the library plasmid DNA as template, Vent DNA polymerase, the plasmid specific primer (e.g., for CITE4, 5′-GATCTGATCTGGGGCCTCGGTGC-3′) (SEQ ID NO:25) and an equimolar preparation of a mixture of primers specific for human JH regions For example:

| | |
|---|---|
| 5′-TGA GGA GAC GGT GAC CAG GGT GCC-3′ | (SEQ ID NO:26); |
| 5′-TGA AGA GAC GGT GAC CAT TGT CCC-3′ | (SEQ ID NO:27); |
| 5′-TGA GGA GAC GGT GAC CAG GGT TCC-3′ | (SEQ ID NO:28); and |
| 5′-TGA GGA GAC GGT GAC CGT GGT CCC-3′ | (SEQ ID NO:29). |

The V_{L} genes are constructed in a separate library as follows.

RNA is prepared as described above. The set of variable light chain primers employed are listed in Table 1 of J D Marks et al, 1991 J. Mol. Biol. 222:581-597, incorporated by reference herein. Reaction mixtures for either the V_{lambda} and Vₖₐₚₚₐ libraries are prepared (50 μL) containing 5 μL of cDNA synthesis product, 20 pmol of back primers, 20 pmol forward primers, 250 μM dNTPs, 10 mM KCl, 10 mM NH4SO4, 20 mM Tris-HCl (pH 8.8), 2 mM MgCl2, 100 μg BSA/mL, 1 U Vent DNA polymerase. Thirty cycles of amplification are used employing a PE Biosystems 9700 GeneAmp instrument. The example cycle parameter is 94° C. for 1 min; 57° C. for 1 min; 72° C. for 1 min.

The PCR products are purified by 2% agarose gel electrophoresis, purified by Geneclean (Bio-101) and dissolved in 25 μL of water. The V_{L} gene repertoire, including the DNA segment encoding the linker segment (e.g. [G4S]3) is amplified from 300 ng of library plasmid DNA by using Vent DNA polymerase, the Gene 3 primer:

| | |
|---|---|
| 5′-GCAAGCCCAATAGGAACCCATGTACCG-3′ | (SEQ ID NO:30); |

and an equimolar mixture of primers for reverse JH-sFv linker segments (e.g., the RHuJ set of Table 1 of J D Marks et al, Id.). The amplified V_{H} and V_{L} genes are purified by agarose electrophoresis and spliced together by overlap extension PCR (Ho SN et al, 1989 Gene 77: 51-59 ) to create the sFv gene repertoire using Vent polymerase.

The V_{H} and V_{L} gene repertoires are spliced together in 100 μL PCR reactions containing 100 ng of the V_{H} and V_{L} DNA fragments and Tth or Vent DNA polymerase for sFv assembly. The reactions are cycled eight times (95° C. for 2 min, 55° C. for 1 min, 72° C. for 3 min) to join the fragments. Next, the CITE4 and Gene3 primers are added and reaction is cycled 30 times (94° C. for 1 min, 55° C. for 1 min, 72° C. for 3 min) to amplify the sFv genes. The sFv genes are digested with suitable restriction enzymes (e.g. NcoI and NotI), purified on agarose gels, and ligated into a suitable plasmid (es pHEN-1). The recombinant plasmids are transformed into a suitable E. coli cell (e.g. TG1).

### Example 2

### Purification of GPIIb/IIIa Antigen

Panning of an sFv phage library for GPIIb/IIIa binders requires a suitable antigen, e.g., a GPIIb/IIIa antigen, for candidate moieties to be screened against. GPIIb/IIIa protein is purified from human platelets as follows.

The mAb A2A9 (A29) (J S Bennett et al., 1983, Proc. Natl. Acad. Sci., (USA) 80:2417-2421) is purified from ascitic fluid with 50% saturated ammonium sulfate. (Alternatively, mAb SSA6 may be employed for immuno-affinity chromatography as described by Weisel et al, Id., supra.) The precipitate is dissolved in a minimal volume of 0.125 M sodium borate buffer, pH 8.4, containing 0.075 M NaCl, dialyzed extensively against this buffer, and subjected to affinity chromatography on protein A-Sepharose (Amersham Pharmacia). Fractions containing anti-platelet antibody activity are eluted from the column with 0.1 M sodium citrate buffer, pH 4.5, and were immediately neutralized with 1 M Tris-HCl, pH 8.5.

Affinity chromatography purification of GPIIb/IIIa protein is performed as described by J S Bennett et al. (above). Purified A29 mAb is coupled to cyanogen bromide-activated Sepharose 4B (1-2 mg of protein per mL of Sepharose) as described by Amersham Pharmacia product literature (cat. no. 17-0430-01). Human platelets, obtained from 250 mL of citrate-anticoagulated blood, are washed free of plasma protein in a buffer containing 150 mM sodium chloride, 14 mM citrate, and 0.7 mM glucose (pH 6.5). The platelets are solubilized in 1% Triton X-100 and the mixture is centrifuged at 250,000×g to remove undissolved debris. The supernatant is passed over a 10 mL of uncoupled resin of Sepharose 4B to remove nonspecific binding material. The flow-through fraction is then applied to a 3 mL column of antibody-Sepharose. Platelet proteins specifically bound to the immobilized antibody are eluted with a buffer containing 0.05 M diethylamine (pH 11.5) and 1% Triton X-100. The eluate is immediately neutralized with 2 M Tris/1% Triton, pH 7.4, and is extensively dialyzed against 0.15 M sodium chloride/0.01 M Tris-HCl, pH 6.8. This provides the purified GPIIb/IIIa preparation that may be buffer-exchanged by diafiltration as needed. Monoclonal antibody SSA6 may be used to pre-coat the microtiter plates prior to the incubation with GPIIb/IIIa antigen.

### Example 3

### Panning of Library for GPIIb/IIIa Binders

Panning of the sFv phage library for GPIIb/IIIa binders is conducted with antigen immobilized on microtiter plate wells, using the protocol 12 as described by Pope et al., Id supra, with minor modifications.

GPIIb/IIIa protein purified, e.g., as described by Example 2, supra, is coated onto plastic microtiter plates (e.g. Nunc Immuno tube Maxisorp 75×12, cat. No.4-44202) with 1 mL of 10 μL/mL GPIIb/IIIa antigen in PBS buffer (10 mM phosphate buffer, pH 7.3, 150 mM NaCl) or 50 mM NaHCO3, pH 9.6). The coated Maxisorp is allowed to stand for about 12 hours at 4° C. or room temperature. The Maxisorp is washed three times with PBS, blocked with 2% (w/v) skimmed milk powder in PBS for 2 h at 37° C. Then the Maxisorp is washed four times with PBS for 2 h at 37° C. Then the Maxisorp is washed four times with PBS. The phage (about 1011 per mL from Example 1) are added to the panning tube as a 1 mL volume. Incubation is done for 1 hour at room temperature or 37° C. The Maxisorp is rinsed 20 times with PBS containing 0.1% Tween-20 and then rinsed 20 times with PBS. Then 1 mL of 100 mM triethylamine is added to the Maxisorp tube and incubation is allowed for 10 min at room temperature. The eluted phage are removed next. 500 μL of 1 M Tris-HCl buffer, pH 7.5, in a 15 mL polypropylene centrifuge tube (Falcon) are added. Half of this eluate (about 750 μL) is added to 5 mL of a mid-log phase culture of E. coli TG1 cells. The cells are shaken slowly at 37° C. for 1 hour. The cells are centrifuged at 3000 g for 5 min and the cells are resuspended in 500 μL 2TYG (Protocol 8 as described by Pope et al., Id., supra. The cells are spread onto 2×TYAG agar plates (100 μL per plate) and grown overnight at 30° C.

After overnight growth, 1.5 mL of 2×TYAGG (2×TYAG with 15% glycerol) are added per plate. The cells are resuspended with a glass plate spreader. The cells are pooled from each plate and aliquoted into Eppendorf tubes. Further rounds of selection are performed by inoculating 25 mL of 2×TYAG with 50 μL of these cells and growing the culture to A600=1.0 before proceeding with phagemid rescue. The selected anti-GPIIb/IIIa clones are analyzed for affinity by standard ELISA and BIAcore methods and for DNA sequence by manual or automated DNA sequencing methods.

### Example 4

### Affinity Maturation of sFv Candidate

The binding affinity of the initial sFv candidate clones is assessed for K_{d} and k_{off} rates as stated above. If affinity improvements are desired, mutagenesis and selection is carried out by procedures described by P S Chowdhury et al, 1999 Nat Biotechnol 17:568-572; R Schier et al, 1996 J. Mol. Biol. 263: 551-567; R Schier et al, J. Mol. Biol. 255:28-43; and K S Johnson and R E Hawkins (Chapter 2 of Antibody Engineering book cited above). The general features of these methods are site-directed or random mutagenesis of the CDR segments; particularly CDR3 of V_{H} and the CDR3 of V_{L} followed by panning under conditions of increasing stringency for selection of higher affinity sFv binders. Where desired, chain shuffling of the variable domains is employed to produce new sets of light and heavy chain pairs.

The general procedure is detailed in the following protocol. Mutagenesis of V_{H} CDR3 or V_{L} CDR3 is performed employing spiked oligonucleotides as described by Johnson and Hawkins, Id. supra. These variant oligonucleotides are selected as complementary strands to the sense strand of the defined CDR segment (Kabat et al., Id. supra) and are readily synthesized on any standard DNA synthesis instrument (e.g. the Applied Biosystems or MilliGen/Biosearch instruments). A range of one to about ten mutations per oligonucleotide are entered at degenerate positions in the primer. The mutations are optionally random or focused on known hot spots for variable chain mutations. For example, a single base change in this oligo will be sufficient to change one codon specification. PCR reactions employing the spiked oligonucleotide plus an upstream fixed primer create the new variable segment. The PCR reaction is set up as follows: combine ten μL of 10×PCR buffer; 5 μL of 4 mM dNTPs; 5 μL of 10 μM reverse primer (e.g. 5′-AGC GGA TAA CAA TTT CAC ACA GG-3′ [SEQ ID NO:31]); 5 μL 10 μM spiked oligo (complementary to CDR3 and framework 4 and containing one to ten base changes in the original CDR3 sequence); 20 ng of template DNA; H₂O to 100 μL; 5 U of Taq DNA polymerase.

PCR amplification is performed using 25 cycles of 94° C. for 1 min, 60° C. for 1 min, and 72° C. for 1 min followed by 10 min at 72° C. The reaction products are purified by agarose electrophoresis of 5 μL on a 1.5% gel. The discrete band at about 350 bp is excised and purified. The light chain segment is amplified in a second PCR reaction by combining 10 μL of 10×PCR buffer; 5 μL of 4 mM dNTPs; 5 μL of 10 μM reverse J_{H} primer

| | |
|---|---|
| (e.g. 5′-GCA CCC TGG TCA CCG TCT CCT CAG GTG G-3′ | [SEQ ID NO:32]); |

5 μl of 10 μM fd gene 3 primer (e.g. 5′-GTC GTC TTT CCA GAC GTT AGT-3′ [SEQ ID NO:33]); 20 ng of template DNA; H₂O to 100 μL; and 5 U of Taq DNA polymerase. Amplification is performed with 25 cycles of 94° C. for 1 min, 60° C. for 1 min, and 72° C. for 1 min, followed by 10 min at 72° C.

Using 1.5% agarose gel electrophoresis, a 400 bp band is identified, excised, and purified. The V_{H} and linker-V_{L} segments are assembled essentially as described in Example 1 to produce intact sFv genes. The variant sFv clones are subjected to stringent panning and analyzed for affinity and DNA sequence as described by Example 3, supra.

### Results

### SCA or sFv Binding Proteins Identified by Screening

Positive clones are identified by the above-methods. The preferred clones encode and produce SCA binding proteins, e.g., sFv binding proteins, that bind to the platelet (or target) antigens employed for the screening process, and that exhibit, relative to their respective target antigens, a K_{d} ranging from about 10⁻M to 10⁻¹² M; a k_{off} ranging from 10⁻³ sec⁻¹to 10⁻⁵ sec⁻¹, and are expressible in E. coli shake-flask cultures with a yield that ranges from about 1 mg/L to 100 mg/L. In addition, the proteins encoded by preferred clones range in molecular weight from about 24 kDa to 30 kDa, as monomers, and multiples of that range when the expressed proteins are present as multimers, e.g., dimers, trimers, and tetramers.

Further, all proteins expressed by preferred clones are sufficiently thermostable at body temperature, i.e., about 37 degrees C., to remain active in vivo, as described by J Willuda et al., 1999 Cancer Res. 59:5758-5767, incorporated by reference herein.

Further still, individual SCA candidates display cross-reactivity to the integrins described in detail, supra, and other individual SCA candidates are highly specific in their binding for only one of these integren-based antigens.

### Example 5

### Production of sFv Proteins by Fermentation in Escherichia coli Host Cells

Fermentation, extraction and purification processes are essentially as described by Filpula et al., 1996, Antibody Engineering, A Practical Approach, Chapter 11 pp 253-256, Id. All of the reagents employed in the following procedures are as described by Filpula, et al., Id. and/or are art-standard, commercially known and available reagents. Alternatively, these processes are conducted as described by Better et al., Id., supra.

A. Preparation of Inoculum

As described by Filpula et al., Id., in Protocol 2, prior to fermentation, an E. coli culture that expresses an sFv binder identified by the above-described screening processes is inoculated into a flask containing 0.5 liters of modified LB medium containing either 50 mg of ampicillin or 25 mg of tetracycline. Typically, a 1 ml vial of frozen E. coli expression strain is employed. The flask is shaken for 10 hours at 32° C.

B. Fermentation

9.5 liters of production medium, i.e., culture medium, as described by Filpula, et al., Id., is inoculated into a Chemap laboratory fermenter (Chemapec, Inc.) together with the 0.5 liters of the inoculum prepared above. The fermentation parameters are adjusted as follows: pH 7.2±0.1 (titrated with 5M NaOH and 2M H₃PO₄); aeration with 1 volume of air per volume medium per minute; agitation at 800 r.p.m. and a temperature of 32° C.

When the absorbance of the production medium reaches 18-20, measured at 600 nm, the fermentation temperature is raised to 42° C. When the sFv expression vector is under the control of a promoter inducible by a chemical additive, the inducer, e.g., IPTG or arabinose is added to the production medium instead of rasing the temperature to 42° C.

At one hour after temperature shift-up, the fermentation is cooled to 10° C., and the cell paste is harvested by centrifugation at 7000 g for 10 minutes (filtration or settling may also be employed for large-scale production). The wet cell paste can be stored at −20° C. The yield is typically about 200-300 g of wet cell paste from a 10 liter fermentation.

In an alternative procedure, the expression vector includes a secretion leader, as described by Better et al. Id., and the cultured E. coli processes the mature protein by secretion to the periplasmic space, followed by leakage of the protein in whole or in part into the production medium.

C. Extraction

When the sFv must be recovered from the cell paste, wherein it is present in an insoluble form, it is readily recovered employing the method described by Filpula et al., Id., Protocol 3. The cell paste is gently resuspended (after thawing at 10° C. overnight if frozen) in 2.5 liters of cell lysis buffer (50 mM Tris-HCl, 1.0 mM EDTA, 100 mM KCl, 0.1 mM phenylmethylsulfonyl fluoride or “PMSF”, pH 8.0) at 4° C. The cell suspension is passed through a Manton-Gaulin cell homogenizer three times, with cooling to bring the temperature back down to 5±2° C., after each pass.

The cell lysate is spun at 24,300 g for 30 minutes at 6° C. and the supernatant discarded. The pellet is resuspended in 1.2 liters of cell lysis buffer, optionally up to 5 times. The washed pellet is then solubilized in freshly prepared denaturing buffer (6 M guanidine hydrochloride, 50 mM Tris-HCl, 10 mM CaCl₂, 50 mM KCl, pH 8.0) at 4° C., using 6 ml/gram of pellet. The suspension is then centrifuged at 24,300 g for 45 minutes at 6° C., and the pellet discarded. If the optical density of the retained supernatant (OD₂₈₀) is above 30, additional denaturing buffer should be added until the OD₂₈₀ reaches about 25.

The supernatant is then slowly diluted about ten-fold into cold (4-10° C.) refolding buffer (50 mM Tris-HCl, 10 mM CaCl₂, 50 mM KCl, 0.1 mM PMSF, pH 8.0), optimally over a 2 hour period, with gentle mixing. The refolding buffer is then allowed to stand undisturbed for at least 20 hours at 4° C.

The solution is then filtered through a 0.45 micron microporous membrane at 4° C. and the filtrate is concentrated to a volume of about 500 ml at 4° C.

D. Cation-Exchange HPLC Purification of sFv Proteins

After cultivation and extraction of crude proteins that include a candidate sFv anti-platelet binding protein, there is a need to obtain a substantially purified sFv product. Since sFv proteins typically have an isolectric point between 8.0 and 9.4, the conjugates of the invention are readily purified by cation exchange chromatography. The sFv source for cation-exchange HPLC purification is the concentrated filtrate produced by the process described in Section C, above, and/or concentrated sFv containing production medium when the sFv is expressed in soluble form, e.g., by a vector comprising a functional secretion leader.

A solution containing soluble sFv protein (or resolubilized sFv protein) is dialyzed against HPLC buffer A (60 mM Mops, 0.5 mM Ca acetate, pH 6.4), until the measured conductivity of the sFv solution is lowered to that of buffer A. A 21.5 mm×150 mm polyaspartic acid PolyCAT A column is equilibrated with HPLC buffer A for 20 minutes. The dialysed sFv sample is loaded on the PolyCAT A column which is run on a Waters BPLC system (Millipore). For this column size, the sample limit is 60 mg for each run.

The protein concentration of the dialysate and eluate is determined by measurement of optical density at 280 nm. Since most sFv proteins have an extinction coefficient of about 2.0 mg ml⁻¹ cm⁻¹ at 280 nm, this is used to calculate the protein concentration from the optical density readings taken at 280 nm.

The sample is eluted from the PolyCAT A column with a 50 minute linear gradient between HPLC buffers A and B (60 mM Mops, 10 mM CaCl₂, pH 7.5), although the gradient can be adjusted for variations in the sFv elution curve. Similarly, fractions are spaced appropriately for the elution curve. Typically 3 minute fractions are acceptable and monomer sFv proteins elute between 20 and 30 minutes with this gradient (multimers elute later).

A final 6 minute linear gradient at 15 ml/min (of 90 mL) is applied to the PolyCAT A column to remove remaining proteins with HPLC buffer C (60 mM Mops, 100 mM CaCl₂, pH 7.5).

The collected fractions are analyzed on 4-20% Tris-glycine SDS-PAGE gels by standard methods. These are readily obtainable from Novex as pre-cast slab gels.

### Example 6

### Tc-99m Diagnostic Imaging of Myocardial Infarction

Imaging Reagent

An anti-platelet binding protein, in the form of an sFv that is identified and produced by the methods described above, is labelled with Tc-99m by any standard art-known method. One convenient method is described, for example, by U.S. Pat. No. 5,328,679 that describes the rapid labeling of a protein with Tc-99m, from reduced pertechetate. This sFv-Tc-99m preparation is used for imaging a number of cardiovascular lesions, such as atherosclerotic plaques, thrombi and emboli.

Cardiac Imaging

A 75 year-old female with a history of recurrent angina is admitted to the emergency room complaining of severe chest pain. The onset of chest pain is stated to be about 2 hours previously. The patient is injected intravenously with 0.25 mg (15 mCi of Tc-99m) of the sFv-Tc-99m conjugate described above, and the patient's heart scanned by single-photon emission computed tomography (SPECT) 30 minutes and again 2 hours later. Both scanning sessions reveal uptake of Tc-99m in the apical region of the left ventricle, with enhanced imaging at the 2-hour scan. Subsequent EKG changes and cardiac enzyme biochemistry suggest an left ventricle infarct. Later studies with thallium indicate a defect in the same area of the heart, confirming the very early detection made by the anti-platelet antigen-binding protein. Later EKG's also show definitive abnormalities consistent with the imaging findings.

Deep Vein Thrombosis Imaging

Mongrel dogs (25-35 lb., fasted overnight) are sedated with a combination of ketamine and aceprozamine intramuscularly and then anesthetized with sodium pentobarbital intravenously. An 18-gauge angiocath is inserted in the distal half of the right femoral vein and an 8 mm Dacron™-entwined stainless steel embolization coil (Cook Co., Bloomington Ind.) is placed in the femoral vein at approximately mid-femur in each animal. The catheter is removed, the wound sutured and the placement of the coil documented by X-ray. The animals are then allowed to recover overnight.

One day following coil placement, each animal is re-anesthetized, intravenous saline drips placed in each foreleg and a urinary bladder catheter inserted to collect urine. The animal is placed supine under a gamma camera which is equipped with a low-energy, all purpose collimator and photopeaked for Tc-99m. Images are acquired on a NucLear Mac computer system.

Tc-99m labeled sFv as described above (5-10 mCi) is injected into one foreleg intravenous line at its point of insertion. The second line is maintained for blood collection. Anterior images over the legs are acquired for 500,000 counts or 20 min (whichever is shorter), at approximately 10-20 min, and at approximately 1, 2, 3 and 4 h post-injection. Following the collection of the final image, each animal is deeply anesthetized with pentobarbital. Two blood samples are collected on a cardiac puncture using a heparinized syringe followed by a euthanasing dose of saturated potassium chloride solution administered by intercardiac or bolus intravenous injection. The femoral vein containing the thrombus and samples of thigh muscle are then carefully dissected out. The thrombus is then dissected free of the vessel and placed in a pre-weighed test tube. The thrombus samples are then weighed and counted in a gamma well counter in the Tc-99m channel. Known fractions of the injected doses are counted as well.

Fresh thrombus weight, percent injected dose (%ID)/g in the thrombus and blood obtained just prior to euthanasia and thrombus/blood and thrombus/muscle ratios are determined. Thrombus/background ratios are determined by analysis of the counts/pixel measured in regions-of-interest (ROI) drawn over the thrombus and adjacent muscle from computer-stored images.

These results confirm that deep vein thrombi can be rapidly and efficiently located in vivo.

While there have been described what are presently believed to be the preferred embodiments of the invention, those skilled in the art will realize that changes and modifications may be made thereto without departing from the spirit of the invention. It is intended to claim all such changes and modifications that fall within the true scope of the invention. Numerous references are cited in the specification, the disclosures of which are incorporated by reference in their entireties.

What is claimed is:

1. A nucleic acid molecule, or its complement, that encodes an anti-platelet binding protein, wherein the nucleic acid is isolated from a phage display library by an in vitro selection process that comprises screening a diverse human antibody variable domain expression library against at least one human platelet antigen, and the human antibody variable domain expression library expresses single-chain proteins.

2. The nucleic acid molecule of claim 1, wherein the human antibody variable domain expression library is derived from human immune cell variable sequences and the in vitro selection process comprises building a human sFv phage display library and panning under conditions that select a nucleic acid molecule encoding an sFv able to bind to at least one human platelet antigen with high affinity.

3. The nucleic acid of claim 1 wherein the platelet antigen is selected from the group consisting of a human platelet glycoprotein IIb, human platelet glycoprotein IIIa, human platelet glycoprotein IIb/IIIa complex, a human platelet alphaᵥbeta₃ vitronectin receptor, a peptide sequence of human leukocyte integrin alpha_{M} I domain ranging from about Cys₁₂₈ to about Ser₁₇₂, a peptide having the amino acid sequence of GCPQEDSDIAFLIDGSGSIIPHDF (SEQ ID NO:1), a peptide having the amino acid sequence of DYPVDIYYLMDLSYSMKDDLWSIQN (SEQ ID NO:2) and combinations thereof.

4. An expression vector comprising the nucleic acid of claim 1 operably linked to a promoter.

5. The expression vector of claim 4 wherein the promoter is inducible.

6. The expression vector of claim 4 wherein the promotor is tissue-specific.

7. The expression vector of claim 6 wherein the promotor is tissue-specific for a tissue selected from the group consisting of arterial endothelial cells, mammary gland cells, bladder epithelium, and combinations thereof.

8. The expression vector of claim 6 wherein the promoter is effective for expression in plant cells.

9. The expression vector of claim 6, wherein said promoter is selected from the group consisting of a beta-lactamase promoter, a lac promoter, a trp promoter, a phoA promoter, an araBAD promoter, a T7 promoter, derivatives of the lambda PL and PR promotor and an OL/PR hybrid promoter.

10. The expression vector of claim 4, selected from the group consisting of a plasmid, a phage, a cosmid, a retrovirus vector, a DNA virus vector and a chromosomally integrated vector.

11. A host cell comprising the vector of claim 4.

12. A non-human mammal comprising cells transformed by the vector of claim 4 that produces the anti-platelet binding protein expressed by the vector.

13. A non-human transgenic mammal that comprises the vector of claim 4 that produces the anti-platelet binding protein expressed by the vector.

14. An anti-platelet binding protein encoded by the nucleic acid molecule of claim 1 that binds to activated or non-activated human platelet glycoprotein IIb/IIIa receptor and inhibits platelet aggregation or thrombus formation.

15. The anti-platelet binding protein of claim 14 that is an sFv protein that has a molecular weight ranging from about 24 kDa to about 30 kDa, per sFv subunit.

16. The anti-platelet binding protein of claim 14 that is an sFv protein that binds to a human platelet glycoprotein IIb/IIIa receptor with a K_{d} ranging from about 1 pM to about 10 nM and a k_{off} from about 10⁻⁵ sec⁻¹ to about 10⁻³ sec⁻¹.

17. A composition comprising the anti-platelet binding protein of claim 14 and a pharmaceutically acceptable carrier.

18. A substantially isolated and purified human antibody that binds to a platelet antigen that comprises an active antigen-binding site of the anti-platelet binding protein of claim 14.

19. The substantially isolated and purified human antibody of claim 18 that is a monoclonal antibody.

20. A human anti-platelet binding protein that comprises a fragment of the human antibody of claim 18 selected from the group consisting of an Fab fragment, an Fv fragment, a light chain fragment, a heavy chain fragment, and combinations thereof.

21. A conjugate comprising a non-antigenic polymer covalently linked to the single-chain antigen-binding polypeptide of claim 15.

22. The conjugate of claim 21, wherein said non-antigenic polymer is selected form the group consisting of dextran, polyvinyl pyrrolidones, polyacryl amides, polyvinyl alcohols and carbohydrate-based polymers.

23. The conjugate of claim 21, wherein said non-antigenic polymer is a polyalkylene oxide.

24. The conjugate of claim 23, wherein said polyalkylene oxide comprises an alkyl terminal.

25. The conjugate of claim 23, wherein said polyalkylene oxide is polyethylene glycol.

26. The conjugate of claim 24, wherein said alkyl-terminated polyalkylene oxide is a monomethyl-terminated polyethylene glycol, (mPEG).

27. The conjugate of claim 21, wherein said non-antigenic polymer has a molecular weight ranging from about 200 to about 60,000.

28. The conjugate of claim 21, wherein said non-antigenic polymer has a molecular weight ranging from about from about 1,000 to about 40,000.

29. The conjugate of claim 21, wherein said non-antigenic polymer has a molecular weight ranging from about 2,000 to about 12,500.

30. The conjugate of claim 21, comprising a thiol linkage between said single-chain antigen-binding polypeptide and said non-antigenic polymer.

31. The conjugate of claim 21, comprising a urethane linkage between said single-chain antigen-binding polypeptide and said non-antigenic polymer.

32. A method of producing an anti-platelet binding protein by culturing a host cell transformed with the vector of claim 4.

33. A method of producing an anti-platelet binding protein by administering the vector of claim 4 to a mammal, under conditions effective to allow cells of said mammal to be productively transfected with said vector.

34. A method of producing an anti-platelet binding protein by producing a transgenic mammal expressing the vector of claim 4 in a tissue selected from the group consisting of arterial endothelial cells, mammary gland, bladder epithelium, and combinations thereof.

35. A method of producing an anti-platelet binding protein by producing a transgenic plant expressing the vector of claim 4 in a plant tissue selected from the group consisting of seeds, fruit, leaves, stems, roots, and combinations thereof.

36. A method of inhibiting platelet aggregation or platelet mediated thrombus formation in blood comprising contacting said blood with an effective amount of the anti-platelet binding protein of claim 14.

37. A method of inhibiting platelet aggregation or platelet mediated thrombus formation in blood comprising contacting said blood with host cells that produce the anti-platelet binding protein of claim 14.

38. The method of claim 36 wherein said blood is present in a mammal.

39. The method of claim 37 wherein said host cells are autologous to said mammal.

40. The method of claim 36 wherein said anti-platelet binding protein is present in said blood in a concentration ranging from about 1 pg to 1 mg per ml of whole blood.

41. A method of inhibiting platelet aggregation or platelet mediated formation of fibrin in a blood vessel, said blood vessel having an endothelial lining in need of treatment thereof, comprising contacting the endothelial lining of said blood vessel with the vector of claim 4.

42. The method of claim 41 wherein said blood vessel is an autologous graft that is contacted with the vector before or after being grafted into a mammal in need thereof.

43. The method of claim 38 wherein said mammal is a human.


SEQUENCE LISTING (33 sequences)

SEQ ID NO: 1 - protein, 24 aa, Human
GCPQEDSDIAFLIDGSGSIIPHDF

SEQ ID NO: 2 - protein, 25 aa, Human
DYPVDIYYLMDLSYSMKDDLWSIQN

SEQ ID NO: 3 - DNA, 32 bp, Mouse
  misc_feature (1)..(32): VH1FOR-2 Primer for variable region
tgaggagacggtgaccgtggtcccttggcccc

SEQ ID NO: 4 - DNA, 22 bp, Mouse
  misc_feature (1)..(22): VH1BACK PRIMER for variable region
aggtsmarctgcagsagtcwgg

SEQ ID NO: 5 - DNA, 24 bp, Mouse
  misc_feature (1)..(24): MJK1FONX Primer for variable region
ccgtttgatttccagcttggtgcc

SEQ ID NO: 6 - DNA, 24 bp, Mouse
  misc_feature (1)..(24): MJK2FONX Primer for variable region
ccgttttatttccagcttggtccc

SEQ ID NO: 7 - DNA, 24 bp, Mouse
  misc_feature (1)..(24): MJK4FONX Primer for variable region
ccgttttatttccaactttgtccc

SEQ ID NO: 8 - DNA, 24 bp, Mouse
  misc_feature (1)..(24): MJK5FONX  Primer for variable region
ccgtttcagctccagcttggtccc

SEQ ID NO: 9 - DNA, 24 bp, Mouse
  misc_feature (1)..(24): VK2BACK Primer for variable region
gacattgagctcacccagtctcca

SEQ ID NO: 10 - DNA, 24 bp, Artificial Sequence
  misc_feature (1)..(24): LINKFOR Primer for insertion of inter-domain linker
tggagactgggtgagctcaatgtc

SEQ ID NO: 11 - DNA, 24 bp, Artificial Sequence
  misc_feature (1)..(24): LINKBACK Primer for insertion of inter-domain linker
gggaccacggtcaccgtctcctca

SEQ ID NO: 12 - DNA, 93 bp, Artificial Sequence
  misc_feature (1)..(93): Primer for insertion of additional interdomain linker
ggcaccacggtcaccgtctcctcaggtggaggcggttcaggcggaggtggctctggcggtggcggatcggacatcgagctcactcagtctcca

SEQ ID NO: 13 - DNA, 56 bp, Artificial Sequence
  misc_feature (1)..(56): VH1BACKSfi Primer for insertion of restriction site
gtcctcgcaactgcggcccagccggccatggcccaggtsmarctgcagsagtcwgg

SEQ ID NO: 14 - DNA, 42 bp, Mouse
  misc_feature (1)..(42): Primer for insertion of restriction site
gagtcattctgcggccgcccgtttgatttccagcttggtgcc

SEQ ID NO: 15 - DNA, 42 bp, Mouse
  misc_feature (1)..(42): JK2NOT10 Primer for insertion of restriction site
gagtcattctgcggccgcccgttttatttccagcttggtccc

SEQ ID NO: 16 - DNA, 42 bp, Mouse
  misc_feature (1)..(42): JK4NOT10 Primer for insertion of restriction site
gagtcattctgcggccgcccgttttatttccaactttgtccc

SEQ ID NO: 17 - DNA, 42 bp, Mouse
  misc_feature (11)..(18): Restriction site
gagtcattctgcggccgcccgtttcagctccagcttggtccc

SEQ ID NO: 18 - DNA, 24 bp, Human
  misc_feature (1)..(24): Primer for IgM specific variable gene mRNA
tggaagaggcacgttcttttcttt

SEQ ID NO: 19 - DNA, 23 bp, Human
  misc_feature (1)..(23): Primer for VH gene
caggtgcagctggtgcagtctgg

SEQ ID NO: 20 - DNA, 23 bp, Human
  misc_feature (1)..(23): Primer for VH gene
caggtcaacttaagggagtctgg

SEQ ID NO: 21 - DNA, 23 bp, Human
  misc_feature (1)..(23): Primer for VH gene
gaggtgcagctggtggagtctgg

SEQ ID NO: 22 - DNA, 23 bp, Human
  misc_feature (1)..(23): Primer for VH gene
caggtgcagctgcaggagtcggg

SEQ ID NO: 23 - DNA, 23 bp, Human
  misc_feature (1)..(23): Primer for VH gene
gaggtgcagctgttgcagtctgc

SEQ ID NO: 24 - DNA, 23 bp, Human
  misc_feature (1)..(23): Primer for VH gene
caggtacagctgcagcagtcagg

SEQ ID NO: 25 - DNA, 23 bp, CITE4
  misc_feature (1)..(23): Primer for plasmid CITE4
gatctgatctggggcctcggtgc

SEQ ID NO: 26 - DNA, 24 bp, Human
  misc_feature (1)..(24): Primer for human JH region
tgaggagacggtgaccagggtgcc

SEQ ID NO: 27 - DNA, 24 bp, Human
  misc_feature (1)..(24): Primer for human JH region
tgaagagacggtgaccattgtccc

SEQ ID NO: 28 - DNA, 24 bp, Human
  misc_feature (1)..(24): Primers for human JH regions
tgaggagacggtgaccagggttcc

SEQ ID NO: 29 - DNA, 24 bp, Human
  misc_feature (1)..(24): Primer for human JH region
tgaggagacggtgaccgtggtccc

SEQ ID NO: 30 - DNA, 27 bp, Bacteriophage fd
  misc_feature (1)..(27): Gene 3 primer
gcaagcccaataggaacccatgtaccg

SEQ ID NO: 31 - DNA, 23 bp, Artificial Sequence
  misc_feature (1)..(23): Primer for mutagenesis of variable region
agcggataacaatttcacacagg

SEQ ID NO: 32 - DNA, 28 bp, Human
  misc_feature (1)..(28): Reverse JH Primer
gcaccctggtcaccgtctcctcaggtgg

SEQ ID NO: 33 - DNA, 21 bp, Bacteriophage fd
  misc_feature (1)..(21): Gene 3 Primer
gtcgtctttccagacgttagt